SEQUENCE LISTING (25 sequences)

SEQ ID NO: 1 - protein, 149 aa, Artificial Sequence
  Synthetic Construct
DSEEDEEHTIITDTELPPLKLMHSFCAFKADDGPCKAIMKRFFFNIFTRQCEEFIYGGCEGNQNRFESLEECKKMCTRDNANRIIKTTLQQEKPDFCFLEEDPGICRGYITRYFYNNQTKQCERFKYGGCLGNMNNFETLEECKNICED

SEQ ID NO: 2 - protein, 254 aa, Artificial Sequence
  Synthetic Construct
DSEEDEEHTIITDTELPPLKLMHSFCAFKADDGPCKAIMKRFFFNIFTRQCEEFIYGGCEGNQNRFESLEECKKMCTRDNANRIIKTTLQQEKPDFCFLEEDPGICRGYITRYFYNNQTKQCERFKYGGCLGNMNNFETLEECKNICEDGPNGFQVDNYGTQLNAVNNSLTPQSTKVPSLFEFHGPSWCLTPADRGLCRANENRFYYNSVIGKCRPFKYSGCGGNENNFTSKQECLRACKKGFIQRISKGGLIK

SEQ ID NO: 3 - protein, 189 aa, Artificial Sequence
  Synthetic Construct
DAAQEPTGNNAEICLLPLDYGPCRALLLRYYYDRYTQSCRQFLYGGCEGNANNFYTWEACDDACWRIEKVPKVCRLQVSVDDQCEGSTEKYFFNLSSMTCEKFFSGGCHRNRIENRFPDEATCMGFCAPKKIPSFCYSPKDEGLCSANVTRYYFNPRYRTCDAFTYTGCGGNDNNFVSREDCKRACAKA

SEQ ID NO: 4 - protein, 146 aa, Artificial Sequence
  Synthetic Construct
LSEEADDTDSELGSMKPLHTFCAMKADDGPCKAMIRSYFFNMYTHQCEEFIYGGCEGNENRFDTLEECKKTCIPGYEKTAVKAASGAERPDFCFLEEDPGLCRGYMKRYLYNNQTKQCERFVYGGCLGNRNNFETLDECKKICENP

SEQ ID NO: 5 - protein, 149 aa, Artificial Sequence
  Synthetic Construct
DSEEDEEYTIITDTELPPLKLMHSFCAFKPDDGPCKAIMKRFFFNIFTRQCEEFIYGGCGGNQNRFESMEECKKVCTRDNVNRIIQTALQKEKPDFCFLEEDPGICRGYITRYFYNNQSKQCERFKYGGCLGNMNNFETLEECKNTCED

SEQ ID NO: 6 - protein, 149 aa, Artificial Sequence
  Synthetic Construct
AAEEDEEFTNITDIKPPLQKPTHSFCAMKVDDGPCRAYIKRFFFNILTHQCEEFIYGGCEGNENRFESLEECKEKCARDYPKMTTKLTFQKGKPDFCFLEEDPGICRGYITRYFYNNQSKQCERFKYGGCLGNLNNFESLEECKNTCEN

SEQ ID NO: 7 - protein, 148 aa, Artificial Sequence
  Synthetic Construct
LPEEDDDTINTDSELRPMKPLHTFCAMKAEDGPCKAMIRSYYFNMNSHQCEEFIYGGCRGNKNRFDTLEECRKTCIPGYKKTTIKTTSGAEKPDFCFLEEDPGICRGFMTRYFYNNQSKQCEQFKYGGCLGNSNNFETLEECRNTCED

SEQ ID NO: 8 - protein, 14 aa, Artificial Sequence
  Synthetic Construct
TGSSSNIGAGYDVH

SEQ ID NO: 9 - protein, 7 aa, Artificial Sequence
  Synthetic Construct
GNSNRPS

SEQ ID NO: 10 - protein, 12 aa, Artificial Sequence
  Synthetic Construct
QSYDSSLSGSGV

SEQ ID NO: 11 - protein, 113 aa, Artificial Sequence
  Synthetic Construct
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGSGVFGGGTKLTVLG

SEQ ID NO: 12 - protein, 218 aa, Artificial Sequence
  Synthetic Construct
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGSGVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 13 - protein, 10 aa, Artificial Sequence
  Synthetic Construct
GFTFSSYAMS

SEQ ID NO: 14 - protein, 17 aa, Artificial Sequence
  Synthetic Construct
AISGSGGSTYYADSVKG

SEQ ID NO: 15 - protein, 11 aa, Artificial Sequence
  Synthetic Construct
LGATSLSAFDI

SEQ ID NO: 16 - protein, 120 aa, Artificial Sequence
  Synthetic Construct
QVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAILGATSLSAFDIWGQGTMVTVSS

SEQ ID NO: 17 - protein, 449 aa, Artificial Sequence
  Synthetic Construct
QVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAILGATSLSAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 18 - protein, 120 aa, Artificial Sequence
  Synthetic Construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAILGATSLSAFDIWGQGTMVTVSS

SEQ ID NO: 19 - protein, 449 aa, Artificial Sequence
  Synthetic Construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAILGATSLSAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 20 - protein, 120 aa, Artificial Sequence
  Synthetic Construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKLGATSLSAFDIWGQGTMVTVSS

SEQ ID NO: 21 - protein, 449 aa, Artificial Sequence
  Synthetic Construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKLGATSLSAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 22 - protein, 212 aa, Artificial Sequence
  Synthetic Construct
DIELTQPPSVSVAPGQTARISCSGDNLRNYYAHWYQQKPGQAPVVVIFYDVNRPSGIPERFSGSNSGNTATLTISGTQAEDEADYYCQSWWDGVPVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 23 - protein, 446 aa, Artificial Sequence
  Synthetic Construct
QVQLVESGGGLVQPGGSLRLSCAASGFTFRSYGMDWVRQAPGKGLEWVSSIRGSRSSTYYADSVKGRFTISRDNSKNTLYLQMNSLRAEDTAVYYCARLYRYWFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 24 - protein, 219 aa, Artificial Sequence
  Synthetic Construct
DIVMTQTPLSLSVTPGQPASISCKSSQSLLESDGKTYLNWYLQKPGQSPQLLIYLVSILDSGVPDRFSGSGSGTDFTLKISRVEAEDVGVYYCLQATHFPQTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 448 aa, Artificial Sequence
  Synthetic Construct
EVQLVESGGGLVKPGGSLRLSCAASGFTFSNYAMSWVRQTPEKRLEWVATISRSGSYSYFPDSVQGRFTISRDNAKNSLYLQMNSLRAEDTAVYYCARLGGYDEGDAMDSWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

(19) United States
(12) Patent Application Publication
(10) Pub. No.: US 2021/0395391 A1
(43) Pub. Date: Dec. 23, 2021

(54) Dosage Regimen for TFPI Antagonists

(71) Applicant: PFIZER INC., New York, NY (US)

(72) Inventors: Tong Zhu, Hopkinton, MA (US); Steven Arkin, Belmont, MA (US); Matthew H. Cardinal, Boston, MA (US); Chay Ngee Lim, Belmont, MA (US); Satyaprakash Nayak, Natick, MA (US)

(21) Appl. No.: 17/283,801

(22) PCT Filed: Oct. 9, 2019
(86) PCT No.: PCT/IB2019/058597

§ 371 Date: Apr. 8, 2021

Related U.S. Application Data
(60) Provisional application No. 62/802,401, filed on Feb. 7, 2019.
(60) Provisional application No. 62/744,481, filed on Oct. 11, 2018.

(51) Int. Cl. C07K 16/38; A61K 45/06; A61P 7/04

(52) U.S. Cl. CPC C07K 16/38; A61K 2039/505; A61P 7/04; A61K 45/06

(57) ABSTRACT

Dosing regimens for the treatment of coagulation disorders using anti-TFPI antibodies are provided. The methods comprises administering to a subject in need there of an initial dose of about 50 mg to 500 mg of an anti-TFPI antibody or antigen-binding fragment thereof.

### CROSS-REFERENCE TO RELATED APPLICATIONS

This application claims the benefit of U.S. Provisional Application Nos. 62/802,401, filed Feb. 7, 2019, and 62/744,481, filed Oct. 11, 2018, which are hereby incorporated by reference in their entirety.

### REFERENCE TO SEQUENCE LISTING

This application is being filed electronically via EFS-Web and includes an electronically submitted sequence listing in .txt format. The .txt file contains a sequence listing entitled “PC72450A_Sequence_Listing_ST25.txt” created on Aug. 12, 2019, and having a size of 42,589 bytes. The sequence listing contained in this .txt file is part of the specification and is hereby incorporated by reference in its entirety.

### FIELD OF THE INVENTION

The present invention relates to dosage regimens of TFPI antagonist antibodies.

### BACKGROUND OF THE INVENTION

Hemophilia A and B are X-linked genetic disorders resulting from functional deficiencies of the plasma proteins Factor VIII (FVIII) or Factor IX (FIX), respectively. Clinical severity of hemophilia is related to the residual level of clotting factor activity. Factor activity of <1% is associated with a severe phenotype, moderate hemophilia is associated with a factor activity of 2%-5% and mild with a factor activity 5%-40%. This application claims the benefit of U.S. Provisional Application Nos. 62/514,242, filed Jun. 2, 2017; and 62/663,082, filed Apr. 26, 2018, which are hereby incorporated by reference here in their entirety.

The standard of care for these disorders is replacement of the missing coagulation factor through intravenous infusions. The replacement factor is commonly a recombinant protein, such as Xyntha (Factor VIII) or BeneFIX (FIX), but plasma derived products of various purity are still in use. Effective prophylactic treatment requires intravenous injection of factor 3-4 times each week, which results in difficulties in compliance and reduced quality of life. The cost of treatment is also expensive due to the complexity of manufacture of coagulation factors. Furthermore, a significant number of patients, up to 32% of patients with severe Hemophilia A, develop neutralizing antibodies to the administered factors, which are seen as foreign proteins by patients who have mutations in these genes. These patients require alternative means of treatment such as the bypass factor, Factor VIIa (NovoSeven).

An alternative approach to therapy is to bypass the need for replacement factors by augmenting the intact extrinsic pathway. Patients with hemophilia have some ability to stop bleeds through their intact extrinsic pathway; however this is not sufficient to shut down major bleeds or to prevent spontaneous bleeds. The extrinsic pathway is insufficient to provide protection because it is rapidly shut down by Tissue Factor Pathway Inhibitor (TFPI).

WO 2017/029583 discloses TFPI antagonist antibodies and uses thereof, and is hereby incorporated by reference in its entirety. There is a significant, unmet need to identify dosing regimens for TFPI antagonist antibodies that provide effective prophylactic protection with reduced frequency of dosing and allowing alternative routes of delivery (e.g., subcutaneous).

### SUMMARY OF THE INVENTION

Disclosed and exemplified herein are dosing regimens for antibodies (and antigen-binding fragments thereof) that bind to the Tissue Factor Pathway Inhibitor (TFPI). Those skilled in the art will recognize, or be able to ascertain using no more than routine experimentation, many equivalents to the specific embodiments of the invention described herein. Such equivalents are intended to be encompassed by the following embodiments (E).

E1. A method of shortening bleeding time, comprising administering to a subject in need thereof an initial dose of about 50 mg to about 500 mg of an antibody or antigen binding fragment thereof that specifically binds to an epitope in Kunitz Domain 2 (K2) of Tissue Factor Pathway Inhibitor (TFPI), wherein said epitope comprises residues Ile105, Arg107, and Leu131, according to the numbering of SEQ ID NO: 2.
E2. A method of treating or preventing a deficiency in blood coagulation or a bleeding disorder, comprising administering to a subject in need thereof an initial dose of about 50 mg to about 500 mg of an antibody or antigen binding fragment thereof that specifically binds to an epitope in Kunitz Domain 2 (K2) of Tissue Factor Pathway Inhibitor (TFPI), wherein said epitope comprises residues Ile105, Arg107, and Leu131, according to the numbering of SEQ ID NO: 2.
E3. A method of treating or preventing hemophilia A, B or C, comprising administering to a subject in need thereof an initial dose of about 50 mg to about 500 mg of an antibody or antigen binding fragment thereof that specifically binds to an epitope in Kunitz Domain 2 (K2) of Tissue Factor Pathway Inhibitor (TFPI), wherein said epitope comprises residues Ile105, Arg107, and Leu131, according to the numbering of SEQ ID NO: 2.
E4. A method of treating or preventing von Willebrand Disease (vWD), comprising administering to a subject in need thereof an initial dose of about 50 mg to about 500 mg of an antibody or antigen binding fragment thereof that specifically binds to an epitope in Kunitz Domain 2 (K2) of Tissue Factor Pathway Inhibitor (TFPI), wherein said epitope comprises residues Ile105, Arg107, and Leu131, according to the numbering of SEQ ID NO: 2.
E5. A method for reducing the activity of TFPI, comprising administering to a subject in need thereof an initial dose of about 50 mg to about 500 mg of an antibody or antigen binding fragment thereof that specifically binds to an epitope in Kunitz Domain 2 (K2) of Tissue Factor Pathway Inhibitor (TFPI), wherein said epitope comprises residues Ile105, Arg107, and Leu131, according to the numbering of SEQ ID NO: 2.
E6. The method as set forth in any one of E1-E5, wherein the initial dose is selected from the group consisting of about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, and about 500 mg.
E7. The method as set forth in any one of E1-E6, wherein the initial dose is selected from the group consisting of 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, about 450 mg, 475 mg, and 500 mg.
E8. The method as set forth in any one of E1-E7, wherein the initial dose is selected from the group consisting of about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg and about 450 mg.
E9. The method as set forth in any one of E1-E8, wherein the initial dose is selected from the group consisting of about 150 mg, about 175 mg, about 200 mg, about 250 mg, about 300 mg, about 350 mg, about 400 mg and about 450 mg.
E10. The method as set forth in any one of E1-E9, wherein the initial dose is about 150 mg.
E11. The method as set forth in any one of E1-E9, wherein the initial dose is about 200 mg.
E12. The method as set forth in any one of E1-E9, wherein the initial dose is about 250 mg.
E13. The method as set forth in any one of E1-E9, wherein the initial dose is about 300 mg.
E14. The method as set forth in any one of E1-E9, wherein the initial dose is 300 mg.
E15. The method as set forth in any one of E1-E9, wherein the initial dose is about 350 mg.
E16. The method as set forth in any one of E1-E9, wherein the initial dose is about 400 mg.
E17. The method as set forth in any one of E1-E9, wherein the initial dose is about 450 mg.
E18. The method as set forth in any one of E1-E16, further comprising administering to the subject one or more subsequent doses of the antibody or antigen binding fragment thereof.
E19. The method as set forth in E18, wherein the one or more subsequent dose is administered in an amount that is about the same, less than or more than the initial dose.
E20. The method as set forth in E19, wherein the one or more subsequent dose is administered in an amount that is about the same as the initial dose.
E21. The method as set forth in any one of E18-E20, wherein either or both of the initial dose or subsequent dose is selected from the group consisting of about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, and about 500 mg.
E22. The method as set forth in any one of E18-E20, wherein either or both of the initial dose or subsequent dose is selected from the group consisting of 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, and 500 mg.
E23. The method as set forth in any one of E18-E22, wherein either or both of the initial dose or subsequent dose is selected from the group consisting of about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg and about 450 mg.
E24. The method as set forth in any one of E18-E23, wherein either or both of the initial dose or subsequent dose is selected from the group consisting of about 150 mg, about 175 mg, about 200 mg, about 250 mg, about 300 mg, about 350 mg, about 400 mg and about 450 mg.
E25. The method as set forth in E24, wherein either or both of the initial dose or subsequent dose is about 150 mg.
E26. The method as set forth in E25, wherein both the initial and the subsequent dose is about 150 mg.
E27. The method as set forth in E24, wherein either or both of the initial dose or subsequent dose is about 200 mg.
E28. The method as set forth in E24, wherein either or both of the initial dose or subsequent dose is about 250 mg.
E29. The method as set forth in E24, wherein either or both of the initial dose or subsequent dose is about 300 mg.
E30. The method as set forth in E29, wherein both the initial dose and subsequent dose is about 300 mg.
E31. The method as set forth in E24, wherein either or both of the initial dose or subsequent dose is about 350 mg.
E32. The method as set forth in E24, wherein either or both of the initial dose or subsequent dose is about 400 mg.
E33. The method as set forth in E24, wherein either or both of the initial dose or subsequent dose is about 450 mg.
E34. The method as set forth in E19, wherein the subsequent dose is administered in an amount that is less than the initial dose.
E35. The method as set forth in E34, wherein the subsequent dose is about two-thirds the initial dose.
E36. The method as set forth in E35, wherein the initial dose is about 450 mg, and the subsequent dose is about 300 mg.
E37. The method as set forth in E35, wherein the initial dose is about 300 mg, and the subsequent dose is about 200 mg.
E38. The method as set forth in E35, wherein the initial dose is about 150 mg, and the subsequent dose is about 100 mg.
E39. The method as set forth in E34, wherein the one or more subsequent dose is about one-half the initial dose.
E40. The method as set forth in E39, wherein the initial dose is about 400 mg, and the subsequent dose is about 200 mg.
E41. The method as set forth in E39, wherein the initial dose is about 300 mg, and the subsequent dose is about 150 mg.
E42. The method as set forth in E39, wherein the initial dose is 300 mg, and the subsequent dose is 150 mg.
E43. The method as set forth in E39, wherein the initial dose is about 200 mg, and the subsequent dose is about 100 mg.
E44. The method as set forth in E30, wherein the initial dose is about 150 mg, and the subsequent dose is about 75 mg.
E45. The method as set forth in E34, wherein the subsequent dose is about one-third the initial dose.
E46. The method as set forth in E45, wherein the initial dose is about 450 mg, and the subsequent dose is about 150 mg.
E47. The method as set forth in E45, wherein the initial dose is about 300 mg, and the subsequent dose is about 100 mg.
E48. The method as set forth in E45, wherein the initial dose is about 150 mg, and the subsequent dose is about 50 mg.
E49. The method as set forth in E34, wherein the initial dose is about 300 mg, and the subsequent dose is about 75 mg.
E50. The method as set forth in E19, wherein the one or more subsequent dose is administered in an amount that is more than the initial dose.
E51. The method as set forth in E50, wherein the one or more subsequent dose is twice the initial dose.
E52. The method as set forth in E51, wherein the initial dose is about 75 mg, and the subsequent dose is about 150 mg.
E53. The method as set forth in E51, wherein the initial dose is about 100 mg, and the subsequent dose is about 200 mg.
E54. The method as set forth in E51, wherein the initial dose is about 125 mg, and the subsequent dose is about 250 mg.
E55. The method as set forth in E51, wherein the initial dose is about 150 mg, and the subsequent dose is about 300 mg.
E56. The method as set forth in E51, wherein the initial dose is about 200 mg, and the subsequent dose is about 400 mg.
E57. The method as set forth in E51, wherein the initial dose is about 225 mg, and the subsequent dose is about 450 mg.
E58. The method as set forth in E50, wherein the initial dose is about 150 mg, and the subsequent dose is increased to about 175 mg, about 200 mg, about 225 mg, about 250 mg, about 300 mg, about 325 mg, about 350 mg, about 375 mg, about 400 mg, about 425 mg, or about 450 mg.
E59. The method as set forth in E50, wherein the initial dose is about 300 mg and the subsequent dose is increased to about 325 mg, about 350 mg, about 375 mg, about 400 mg, about 425 mg, or about 450 mg.
E60. The method as set forth in any one of E18-E59, wherein the one or more subsequent dose is administered once daily, once every 3 days, once every 6 days, twice a week, once a week (weekly), once every 2 weeks, once every 3 weeks, once every 4 weeks, once every 5 weeks, once every 6 weeks, once every 7 weeks or once every 8 weeks after the initial dose.
E61. The method as set forth in any one of E18-E60, wherein the one or more subsequent dose is administered about 1 week, 2 weeks, 3 weeks, or 4 weeks after the initial dose.
E62. The method as set forth in E61, wherein the one or more subsequent dose is administered 1 week after the initial dose.
E63. The method as set forth in E61, wherein the one or more subsequent dose is administered once a week (weekly) after the initial dose.
E64. The method as set forth in any one of E61, E62 or E63, wherein the initial and subsequent dose is about 150 mg.
E65. The method as set forth in any one of E61, E62 or E63, wherein the initial and subsequent dose is about 300 mg.
E66. The method as set forth in any one of E61, E62 or E63, wherein the initial dose is about 300 mg and the one or more subsequent dose is about 150 mg.
E67. The method as set forth in any one of E61, E62 or E63, wherein the initial dose is 300 mg and the one or more subsequent dose is 150 mg.
E68. The method as set forth in any one of E61, E62 or E63, wherein the initial and subsequent dose is about 450 mg.
E69. The method as set forth in any one of E61, E62 or E63, wherein the initial dose is about 150 mg, and the subsequent dose is increased to about 175 mg, about 200 mg, about 225 mg, about 250 mg, about 300 mg, about 325 mg, about 350 mg, about 375 mg, about 400 mg, about 425 mg, or about 450 mg.
E70. The method as set forth in any one of E61, E62 or E63, wherein the initial dose is about 150 mg and the subsequent dose is increased to about 300 mg.
E71. The method as set forth in any one of E61, E62 or E63, wherein the initial dose is about 300 mg, and the subsequent dose is increased to about 325 mg, about 350 mg, about 375 mg, about 400 mg, about 425 mg, or about 450 mg.
E72. The method as set forth in E71, wherein the initial dose is about 300 mg, and the subsequent dose is increased to about 400 mg.
E73. The method as set forth in E71, wherein the initial dose is about 300 mg, and the subsequent dose is increased to about 425 mg.
E74. The method as set forth in E71, wherein the initial dose is about 300 mg, and the subsequent dose is increased to about 450 mg.
E75. The method as set forth in any one of E1-E74, wherein the antibody or antigen binding fragment thereof is administered subcutaneously.
E76. The method as set forth in any one of E1-E74, wherein the antibody or antigen binding fragment thereof is administered intravenously or intramuscularly.
E77. The method as set forth in any one of E1-E76, wherein the antibody, or antigen-binding fragment thereof, does not bind to Kunitz Domain 1 (K1) of TFPI.
E78. The method as set forth in any one of E1-E77, wherein said epitope further comprises residues Cys106, Gly108, Cys130, Leu131, and Gly132, according to the numbering of SEQ ID NO: 2.
E79. The method as set forth in any one of E1-E78, wherein said epitope further comprises Asp102, Arg112, Tyr127, Gly129, Met134, and Glu138, according to the numbering of SEQ ID NO: 2.
E80. The method as set forth in any one of E1-E79, wherein said epitope does not comprise: E100, E101, P103, Y109, T111, Y113, F114, N116, Q118, Q121, C122, E123, R124, F125, K126, and L140, according to the numbering of SEQ ID NO: 2.
E81. The method as set forth in any one of E1-E80, wherein said epitope does not comprise: D31, D32, P34, C35, K36, E100, E101, P103, Y109, K126, and G128, according to the numbering of SEQ ID NO: 2.
E82. The method as set forth in any one of E1-E81, wherein the antibody or antigen binding fragment thereof comprises a heavy chain variable region (VH) comprising:
(a) a VH complementarity determining region one (CDR-H1) comprising the amino acid sequence of SEQ ID NO: 13;
(b) a VH complementarity determining region two (CDR-H2) comprising the amino acid sequence of SEQ ID NO: 14; and
(c) a VH complementarity determining region three (CDR-H3) comprising the amino acid sequence of SEQ ID NO: 15.
E83. The method as set forth in any one of E1-E82, wherein the antibody or antigen binding fragment thereof comprises a VH comprising an amino acid sequence at least 90%, at least 95%, or at least 99% identical to an amino acid sequence selected from the group consisting of SEQ ID NOs: 16, 18, and 20.
E84. The method as set forth in any one of E1-E83, wherein the antibody or antigen binding fragment thereof comprises a VH comprising an amino acid sequence selected from the group consisting of SEQ ID NOs: 16, 18, and 20.
E85. The method as set forth in any one of E1-E84, wherein the antibody or antigen binding fragment thereof comprises a VH comprising the amino acid sequence of SEQ ID NO: 16.
E86. The method as set forth in any one of E1-E84, wherein the antibody or antigen binding fragment thereof comprises a VH comprising the amino acid sequence of SEQ ID NO: 18.
E87. The method as set forth in any one of E1-E84, wherein the antibody or antigen binding fragment thereof comprises a VH comprising the amino acid sequence of SEQ ID NO: 20.
E88. The method as set forth in any one of E1-E87, wherein the antibody or antigen binding fragment thereof comprises a light chain variable region (VL) comprising:
(a) a VL complementarity determining region one (CDR-L1) comprising the amino acid sequence of SEQ ID NO: 8;
(b) a VL complementarity determining region two (CDR-L2) comprising the amino acid sequence of SEQ ID NO: 9; and
(c) a VL complementarity determining region three (CDR-L3) comprising the amino acid sequence of SEQ ID NO: 10.
E89. The method as set forth in any one of E1-E88, wherein the antibody or antigen binding fragment thereof comprises a VL comprising an amino acid sequence at least 90%, at least 95%, or at least 99% identical to SEQ ID NO: 11.
E90. The method as set forth in any one of E1-E89, wherein the antibody or antigen binding fragment thereof comprises a VL comprising the amino acid sequence of SEQ ID NO: 11.
E91. The method as set forth in any one of E1-E90, wherein the antibody or antigen binding fragment thereof comprises a heavy chain comprising the amino acid sequence of SEQ ID NO: 17.
E92. The method as set forth in any one of E1-E90, wherein the antibody or antigen binding fragment thereof comprises a heavy chain comprising the amino acid sequence of SEQ ID NO: 19.
E93. The method as set forth in any one of E1-E90, wherein the antibody or antigen binding fragment thereof comprises a heavy chain comprising the amino acid sequence of SEQ ID NO: 21.
E94. The method as set forth in any one of E1-E93, wherein the antibody or antigen binding fragment thereof comprises a light chain comprising the amino acid sequence of SEQ ID NO: 12.
E95. The method as set forth in any one of E1-E81, wherein the antibody or antigen binding fragment thereof comprises:
(i) a heavy chain variable region (VH) comprising:
   (a) a VH complementarity determining region one (CDR-H1) comprising the amino acid sequence of SEQ ID NO: 13;
   (b) a VH complementarity determining region two (CDR-H2) comprising the amino acid sequence of SEQ ID NO: 14; and
   (c) a VH complementarity determining region three (CDR-H3) comprising the amino acid sequence of SEQ ID NO: 15, and
(ii) a light chain variable region (VL) comprising:
   (a) a VL complementarity determining region one (CDR-L1) comprising the amino acid sequence of SEQ ID NO: 8;
   (b) a VL complementarity determining region two (CDR-L2) comprising the amino acid sequence of SEQ ID NO: 9; and
   (c) a VL complementarity determining region three (CDR-L3) comprising the amino acid sequence of SEQ ID NO: 10.
      E96. The method as set forth in E95, wherein the antibody or antigen binding fragment thereof comprises a VH comprising the amino acid sequence of SEQ ID NO: 18, and a VL comprising the amino acid sequence of SEQ ID NO: 11.
      E97. The method as set forth in E96, wherein the antibody or antigen binding fragment thereof comprises the VH sequence encoded by the insert present in the plasmid deposited under ATCC Accession No. PTA-122329.
      E98. The method as set forth in E96 or E97, wherein the antibody or antigen binding fragment thereof comprises the VL sequence encoded by the insert present in the plasmid deposited under ATCC Accession No. PTA-122328.
      E99. The method as set forth in E96, wherein the antibody or antigen binding fragment thereof comprises a heavy chain comprising the amino acid sequence of SEQ ID NO: 19, and comprises a light chain comprising the amino acid sequence of SEQ ID NO: 12.
      E100. The method as set forth in E95, wherein the antibody or antigen binding fragment thereof comprises a VH comprising the amino acid sequence of SEQ ID NO: 16, and a VL comprising the amino acid sequence of SEQ ID NO: 11.
      E101. The method as set forth in E100, wherein the antibody or antigen binding fragment thereof comprises a heavy chain comprising the amino acid sequence of SEQ ID NO: 17, and comprises a light chain comprising the amino acid sequence of SEQ ID NO: 12.
      E102. The method as set forth in E95, wherein the antibody or antigen binding fragment thereof comprises a VH comprising the amino acid sequence of SEQ ID NO: 20, and a VL comprising the amino acid sequence of SEQ ID NO: 11.
      E103. The method as set forth in E102, wherein the antibody or antigen binding fragment thereof comprises a heavy chain comprising the amino acid sequence of SEQ ID NO: 21, and comprises a light chain comprising the amino acid sequence of SEQ ID NO: 12.
      E104. The method as set forth in any one of E1-E76, wherein the antibody or antigen binding fragment thereof comprises a heavy chain comprising the amino acid sequence of SEQ ID NO: 23, and comprises a light chain comprising the amino acid sequence of SEQ ID NO: 22.
      E105. The method as set forth in any one of E1-E76, wherein the antibody or antigen binding fragment thereof comprises a heavy chain comprising the amino acid sequence of SEQ ID NO: 25, and comprises a light chain comprising the amino acid sequence of SEQ ID NO: 24.
      E106. The method as set forth in any one of E1-E103, wherein the antibody or antigen binding fragment thereof has a serum half-life of at least 25 hours, at least 29 hours, at least 30 hours at least 35 hours, at least 40 hours, at least 50 hours, at least 55 hours, at least 60 hours, at least 65 hours, at least 70 hours, at least 75 hours, at least 80 hours, at least 85 hours, at least 90 hours, at least 95 hours, at least 100 hours, at least 105 hours, at least 110 hours, at least 115 hours, at least 120 hours or at least 125 hours.
      E107. The method as set forth in any one of E1-E103, wherein the antibody or antigen binding fragment thereof has a binding affinity (K_{D}) of from about 5×10⁻⁷M to about 5×10⁻¹¹ M.
      E108. The method as set forth in any one of E1-E107, wherein the antibody or antigen binding fragment thereof has a subcutaneous (SC) bioavailability of at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90%, at least 95, or at least 99% relative to intravenous bioavailability.
      E109. The method as set forth in any one of E1-E108, wherein the subject suffers from or is susceptible to a deficiency in blood coagulation or a bleeding disorder.
      E110. The method as set forth in any one of E1-E109, wherein the subject suffers from or is susceptible to hemophilia A, B or C.
      E111. The method as set forth in any one of E1-E110, wherein the subject suffers from or is susceptible to hemophilia A or B.
      E112. The method as set forth in any one of E1-E109, wherein the subject suffers from or is susceptible to von Willebrand Disease (vWD).
      E113. The method as set forth in any one of E1-E109, wherein the subject suffers from or is susceptible to a platelet disorder.
      E114. The method as set forth in any one of E1-E109, wherein the subject suffers from or is susceptible to a Factor VII deficiency.
      E115. The method as set forth in any one of E1-E109, wherein the subject suffers from or is susceptible to a Factor XI deficiency.
      E116. The method as set forth in any one of E1-115, wherein administration of the antibody or antigen binding fragment thereof: (i) decreases clotting time as measured in a plasma based dilute prothrombin time (dPT) assay; (ii) reduces clotting time in whole blood as measured by thromboelastrography or rotational thromboelastometry; (iii) increases thrombin generation; (iv) increases FXa activity in the presence of TFPI; (v) enhances platelet accumulation in the presence of TFPI; (vi) increases fibrin generation in the presence of TFPI as measured by D-dimers; (vii) increases level of prothrombin fragment 1+2 (PF1+2); or (viii) any combination thereof.
      E117. The method as set forth in E116, wherein the reduction in clotting time in whole blood is determined using whole blood obtained from a human subject having hemophilia A or B.
      E118. The method as set forth in E117, wherein the reduction in clotting time in whole blood is determined using whole blood obtained from a human subject having (i) hemophilia A and inhibitory antibodies against human Factor VIII or (ii) hemophilia B and inhibitory antibodies against human Factor IX.
      E119. The method as set forth in E116, wherein the reduction in clotting time as measured in a dPT assay is determined using plasma obtained from a human subject having hemophilia A or B.
      E120. The method as set forth in E119, wherein the reduction in clotting time as measured in a dPT assay is determined using plasma obtained from a human subject having (i) hemophilia A and inhibitory antibodies against human Factor VIII or (ii) hemophilia B and inhibitory antibodies against human Factor IX.
      E121. The method as set forth in E116, wherein the increase in thrombin generation is determined using plasma obtained from a human subject having hemophilia A or B.
      E122. The method as set forth in E121, wherein the increase in thrombin generation is determined using plasma obtained from a human subject having (i) hemophilia A and inhibitory antibodies against human Factor VIII or (ii) hemophilia B and inhibitory antibodies against human Factor IX.
      E123. The method as set forth in any of the preceding embodiments, wherein administration of the antibody or antigen binding fragment thereof is sufficient to achieve at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 90%, at least 95%, or at least 99% of normal hemostatic activity.
      E124. The method as set forth in any of the preceding embodiments, wherein administration of the antibody or antigen-binding fragment thereof provides a reduction of at least 20%, at least 25%, at least 30%, at least 35%, at least 40%, at least 45%, at least 50%, at least 55%, at least 60%, at least 65%, at least 70%, at least 75%, at least 80%, at least 90%, at least 95%, at least 96%, at least 98%, or at least 99% in annualized bleeding rate (ABR) as compared to ABR observed in control subjects that have coagulation disorders.
      E125. The method as set forth in E124, wherein the reduction in ABR is compared to control subjects that have hemophilia (e.g., hemophilia A or B).
      E126. The method as set forth in E125, wherein the control subjects have been treated with coagulation replacement therapy on demand (i.e., as needed to treat sudden hemorrhages).
      E127. The method as set forth in any of the preceding embodiments, wherein administration of the antibody or antigen-binding fragment thereof provides a reduction of at least 20%, at least 25%, at least 30%, at least 35%, at least 40%, at least 45%, at least 50%, at least 55%, at least 60%, at least 65%, at least 70%, at least 75%, at least 80%, at least 90%, at least 95%, at least 96%, at least 98%, or at least 99% in annualized bleeding rate (ABR) as compared to the ABR in said subject before administration.
      E128. The method as set forth in any of the preceding embodiments, further comprising administering a clotting agent to the subject.
      E129. The method as set forth in E128, wherein the clotting agent is selected from the group consisting of factor VIIa, factor VIII, factor IX, tranexamic acid and bypass agents (e.g., FEIBA).
      E130. A method of shortening bleeding time, comprising administering to a subject in need thereof an initial dose of 300 mg of an antibody or antigen binding fragment thereof that specifically binds to an epitope in Kunitz Domain 2 (K2) of Tissue Factor Pathway Inhibitor (TFPI), followed by administration of a subsequent dose of 150 mg of the antibody or antigen binding fragment thereof, wherein the subsequent dose is administered once a week (weekly) and wherein the antibody comprises (i) a heavy chain comprising the amino acid sequence of SEQ ID NO: 19, and (ii) a light chain comprising the amino acid sequence of SEQ ID NO: 12.
      E131. A method for treating hemophilia (e.g., hemophilia A or B), comprising administering to a subject in need thereof an initial dose of 300 mg of an antibody or antigen binding fragment thereof that specifically binds to an epitope in Kunitz Domain 2 (K2) of Tissue Factor Pathway Inhibitor (TFPI), followed by administration of a subsequent dose of 150 mg of the antibody or antigen binding fragment thereof, wherein the subsequent dose is administered once a week (weekly) and wherein the antibody comprises (i) a heavy chain comprising the amino acid sequence of SEQ ID NO: 19, and (ii) a light chain comprising the amino acid sequence of SEQ ID NO: 12.
      E132. A method for treating von Willebrand Disease (vWD), comprising administering to a subject in need thereof an initial dose of 300 mg of an antibody or antigen binding fragment thereof that specifically binds to an epitope in Kunitz Domain 2 (K2) of Tissue Factor Pathway Inhibitor (TFPI), followed by administration of a subsequent dose of 150 mg of the antibody or antigen binding fragment thereof, wherein the subsequent dose is administered once a week (weekly) and wherein the antibody comprises (i) a heavy chain comprising the amino acid sequence of SEQ ID NO: 19, and (ii) a light chain comprising the amino acid sequence of SEQ ID NO: 12.
      E133. A method of shortening bleeding time, comprising administering to a subject in need thereof a weekly (once a week) dose of 300 mg of an antibody or antigen binding fragment thereof that specifically binds to an epitope in Kunitz Domain 2 (K2) of Tissue Factor Pathway Inhibitor (TFPI), wherein the antibody comprises (i) a heavy chain comprising the amino acid sequence of SEQ ID NO: 19, and (ii) a light chain comprising the amino acid sequence of SEQ ID NO: 12.
      E134. A method for treating hemophilia (e.g., hemophilia A or B), comprising administering to a subject in need thereof a weekly (once a week) dose of 300 mg of an antibody or antigen binding fragment thereof that specifically binds to an epitope in Kunitz Domain 2 (K2) of Tissue Factor Pathway Inhibitor (TFPI), wherein the antibody comprises (i) a heavy chain comprising the amino acid sequence of SEQ ID NO: 19, and (ii) a light chain comprising the amino acid sequence of SEQ ID NO: 12.
      E135. A method for treating von Willebrand Disease (vWD), comprising administering to a subject in need thereof a weekly (once a week) dose of 300 mg of an antibody or antigen binding fragment thereof that specifically binds to an epitope in Kunitz Domain 2 (K2) of Tissue Factor Pathway Inhibitor (TFPI), wherein the antibody comprises (i) a heavy chain comprising the amino acid sequence of SEQ ID NO: 19, and (ii) a light chain comprising the amino acid sequence of SEQ ID NO: 12.
      E136. A method of reducing annualized bleeding rate (ABR) in a hemophilia subject in need thereof, said method comprising administering a therapeutically effective amount of a TFPI antagonist antibody, wherein the ABR after administration is reduced by at least 75% compared to the ABR in said subject before administration.
      E137. A method of reducing annualized bleeding rate (ABR) in a hemophilia subject in need thereof, said method comprising administering a therapeutically effective amount of a TFPI antagonist antibody, wherein the ABR after administration is reduced by at least 80% compared to the ABR in said subject before administration.
      E138. A method of reducing annualized bleeding rate (ABR) in a hemophilia subject in need thereof, said method comprising administering a therapeutically effective amount of a TFPI antagonist antibody, wherein the ABR after administration is reduced by at least 90% compared to the ABR in said subject before administration.
      E139. A method of reducing annualized bleeding rate (ABR) in a hemophilia subject in need thereof, said method comprising administering a therapeutically effective amount of a TFPI antagonist antibody, wherein the ABR after administration is reduced by at least 95% compared to the ABR in said subject before administration.
      E140. The method as set forth in E136, wherein the percent reduction in ABR after administration compared with the ABR in said subject before administration is selected from the group consisting of:

a) 82% ABR reduction compared to the ABR before administration of said antibody to said subject and wherein the amount is 300 mg administered on a recurring basis;

b) 90% ABR reduction compared to the ABR before administration of said antibody to said subject and wherein the amount is 300 mg followed by 150 mg administered on a recurring basis;

c) 80% ABR reduction compared to the ABR before administration of said antibody to said subject and wherein the amount is 450 mg administered on a recurring basis; and

d) 96% ABR reduction compared to the ABR before administration of said antibody to said subject, wherein the amount is 300 mg administered on a recurring basis, and wherein the subject has hemophilia A and inhibitory antibodies against human Factor VIII or has hemophilia B and inhibitory antibodies against human Factor IX.

E141. The method as set forth in E140, wherein the recurring basis is once per week (QW).
E142. The method as set forth in E140, wherein the recurring basis is once every two weeks.
E143. The method as set forth in E140, wherein the recurring basis is once a day (i.e., daily).
E144. The method as set forth in any one of E136-E143, wherein the antibody is administered subcutaneously (SC) or intravenously (IV).
E145. The method as set forth in E144, wherein the antibody is administered SC.
E146. The method as set forth in E140(a), wherein the mean ABR before administration is at least 23 bleeds per year and the ABR after administration is not more than 4.2 bleeds per year.
E147. The method as set forth in E140(b), wherein the mean ABR before administration is at least 14 bleeds per year and the ABR after administration is not more than 1.5 bleeds per year.
E148. The method as set forth in E140(c), wherein the mean ABR before administration is at least 20 bleeds per year and the ABR after administration is not more than 4.2 bleeds per year.
E149. The method as set forth in E140(d), wherein the mean ABR before administration is at least 17 bleeds per year and the ABR after administration is not more than 0.72 bleeds per year.
E150. A method of reducing annualized bleeding rate (ABR) in a hemophilia subject in need thereof, said method comprising administering a therapeutically effective amount of a TFPI antagonist antibody, wherein the ABR after administration is reduced by at least 75% compared to an ABR historical standard.
E151. A method of reducing annualized bleeding rate (ABR) in a hemophilia subject in need thereof, said method comprising administering a therapeutically effective amount of a TFPI antagonist antibody, wherein the ABR after administration is reduced by at least 80% compared to an ABR historical standard.
E152. A method of reducing annualized bleeding rate (ABR) in a hemophilia subject in need thereof, said method comprising administering a therapeutically effective amount of a TFPI antagonist antibody, wherein the ABR after administration is reduced by at least 90% compared to an ABR historical standard.
E153. The method as set forth in any one of E150-E152, wherein the percent reduction in ABR after administration compared with the historical standard ABR is selected from the group consisting of:

a) 85% ABR reduction compared to the historical standard ABR and wherein the amount is 300 mg administered on a recurring basis;

b) 95% ABR reduction compared to the historical standard ABR and wherein the amount is 300 mg followed by 150 mg administered on a recurring basis; and

c) 85% ABR reduction compared to the historical standard ABR and wherein the amount is 450 mg administered on a recurring basis; and

d) 98% ABR reduction compared to the historical standard ABR, wherein the amount is 300 mg administered on a recurring basis and wherein the subject has hemophilia A and inhibitory antibodies against human Factor VIII or has hemophilia B and inhibitory antibodies against human Factor IX.

E154. The method as set forth in any one of E150-E153, wherein the historical standard ABR is at least 27 bleeds per year.
E155. The method as set forth in any one of E153-E154, wherein the recurring basis is once per week (QW).
E156. The method as set forth in any one of E153-E154, wherein the recurring basis is once every two weeks.
E157. The method as set forth in any one of E153-E154, wherein the recurring basis is daily.
E158. The method as set forth in any one of E150-E157, wherein the antibody is administered subcutaneously (SC) or intravenously (IV).
E159. The method as set forth in E158, wherein the antibody is administered SC.
E160. The method as set forth in any one of E139-E157, wherein the antibody is selected from the group consisting of TFPI-23, TFPI-106, TFPI-107, concizumab (i.e., hz4F36), 2A8 and 2A8-200.
E161. The method as set forth in any one of E139-E158, wherein the antibody comprises a CDR-H1 comprising the amino acid sequence of SEQ ID NO: 13, a CDR-H2 comprising the amino acid sequence of SEQ ID NO: 14, a CDR-H3 comprising the amino acid sequence of SEQ ID NO: 15, a CDR-L1 comprising the amino acid sequence of SEQ ID NO: 8, a CDR-L2 comprising the amino acid sequence of SEQ ID NO: 9, and a CDR-L3 comprising the amino acid sequence of SEQ ID NO: 10.
E162. Use of an antibody or antigen binding fragment thereof that specifically binds to an epitope in Kunitz Domain 2 (K2) of Tissue Factor Pathway Inhibitor (TFPI) in a method of the invention, as set forth in any one of the preceding embodiments.
E163. An antibody or antigen binding fragment thereof that specifically binds to an epitope in Kunitz Domain 2 (K2) of Tissue Factor Pathway Inhibitor (TFPI) for use as set forth in any one of the preceding embodiments.
E164. An antibody or antigen binding fragment thereof that specifically binds to an epitope in Kunitz Domain 2 (K2) of Tissue Factor Pathway Inhibitor (TFPI) in manufacture of a medicament for use in a method as set forth in any one of the preceding embodiments.
E165. An antibody or antigen binding fragment thereof that specifically binds to an epitope in Kunitz Domain 2 (K2) of Tissue Factor Pathway Inhibitor (TFPI) for use in the treatment or prevention of bleeding or a bleeding disorder or a deficiency in blood coagulation, wherein the antibody or antigen binding fragment thereof is administered in an initial dose of 300 mg, followed by administration of a subsequent dose of 150 mg, wherein the subsequent dose is administered once a week (weekly), and wherein the antibody comprises (i) a heavy chain comprising the amino acid sequence of SEQ ID NO: 19, and (ii) a light chain comprising the amino acid sequence of SEQ ID NO: 12.
E166. An antibody or antigen binding fragment thereof for use as set forth in E164, whereby bleeding time is shortened.
E167. An antibody or antigen binding fragment thereof for use according to any one of E165-E166, wherein the antibody or antigen binding fragment thereof is for co-administration or simultaneous, separate or sequential administration with a clotting agent.

### BRIEF DESCRIPTION OF THE DRAWINGS

FIG. 1 is a schematic depicting the study design of the present study (B7841002), an open-label investigation of the safety, tolerability, pharmacokinetics (PK), pharmacodynamics (PD), and efficacy of multiple subcutaneous (SC) doses of TFPI-106 in males with severe hemophilia A or B.

FIG. 2 is a graphic presentation showing the observed plasma concentrations of TFPI-106 overlaid with model predictions of median and 90% prediction interval (PI; shown in gray) for 300 mg SC QW (Cohort 1) (FIG. 2A), 150 mg SC QW with 300 mg loading dose (Cohort 2) (FIG. 2B) and 450 mg SC QW (Cohort 3) (FIG. 2C).

FIG. 3 depicts the median values for Total TFPI (FIG. 3A), TGA peak thrombin (FIG. 3B), or median change from baseline for PF1+2 (FIG. 3C), dPT (FIG. 3D) and D-dimer (FIG. 3E) vs. time by dose cohort.

### DETAILED DESCRIPTION OF THE INVENTION

### 1. Overview

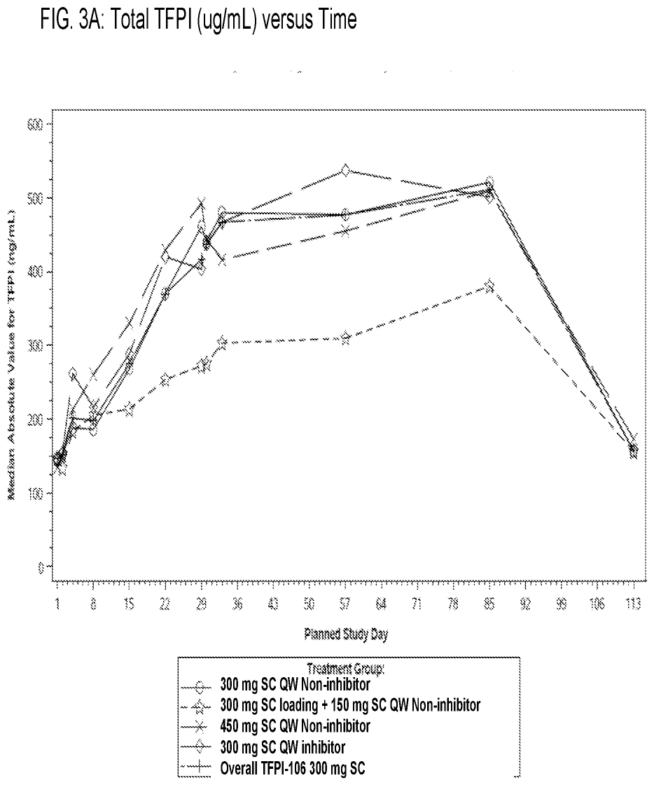

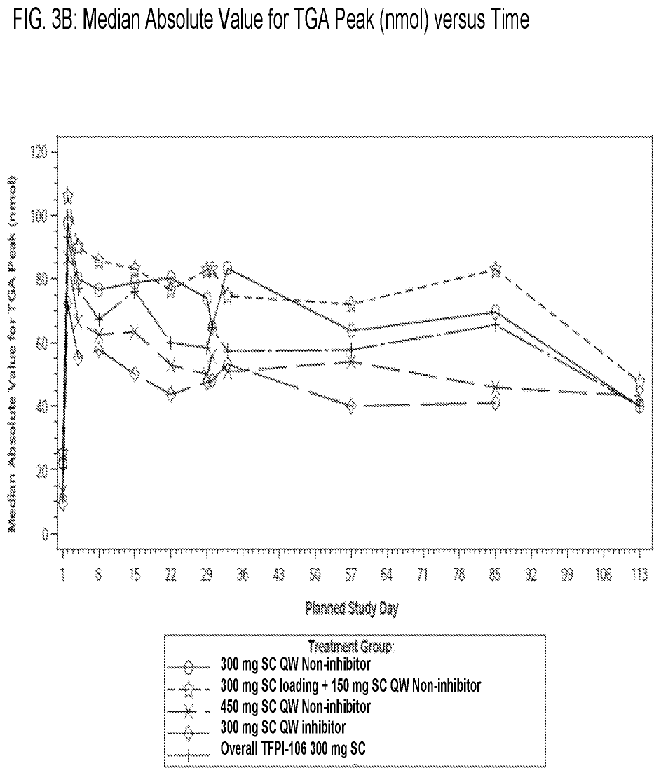

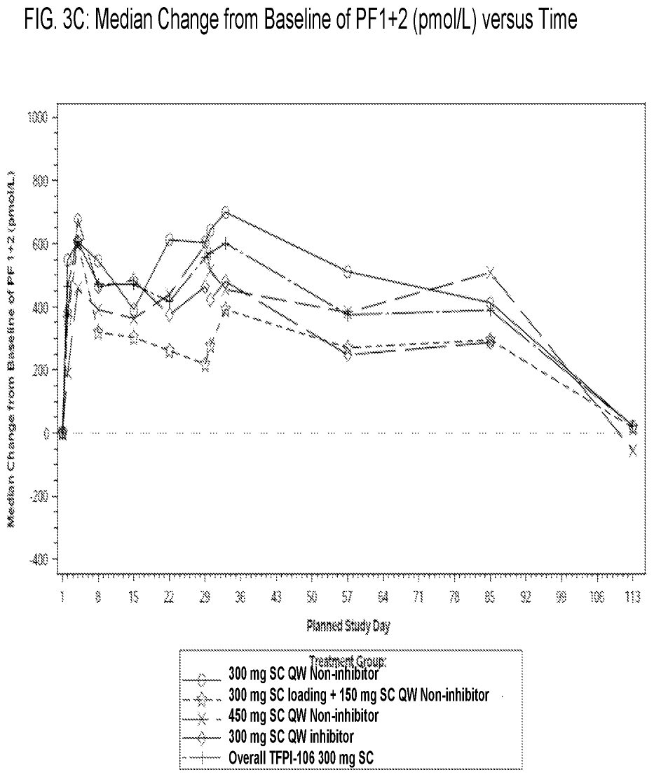

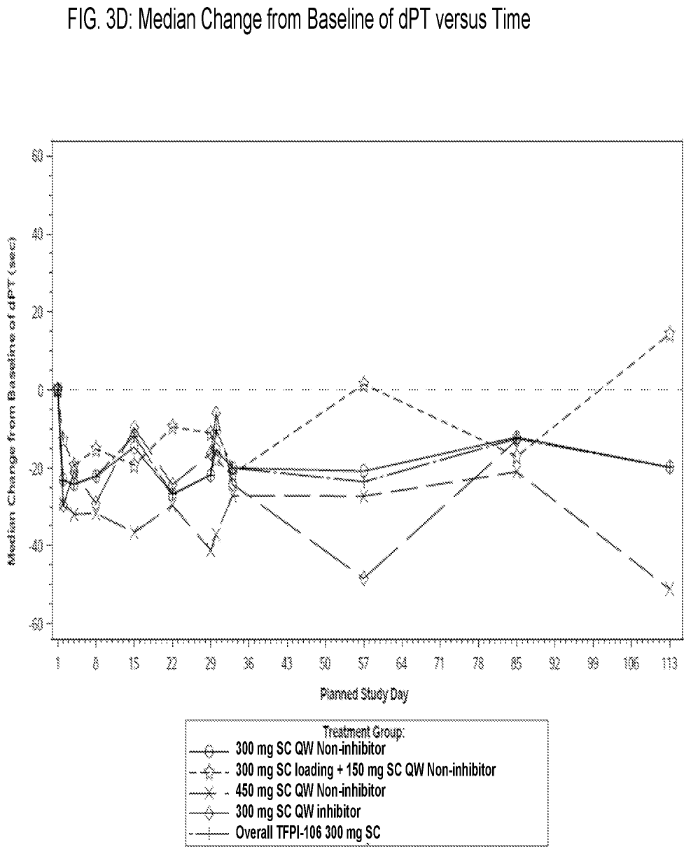

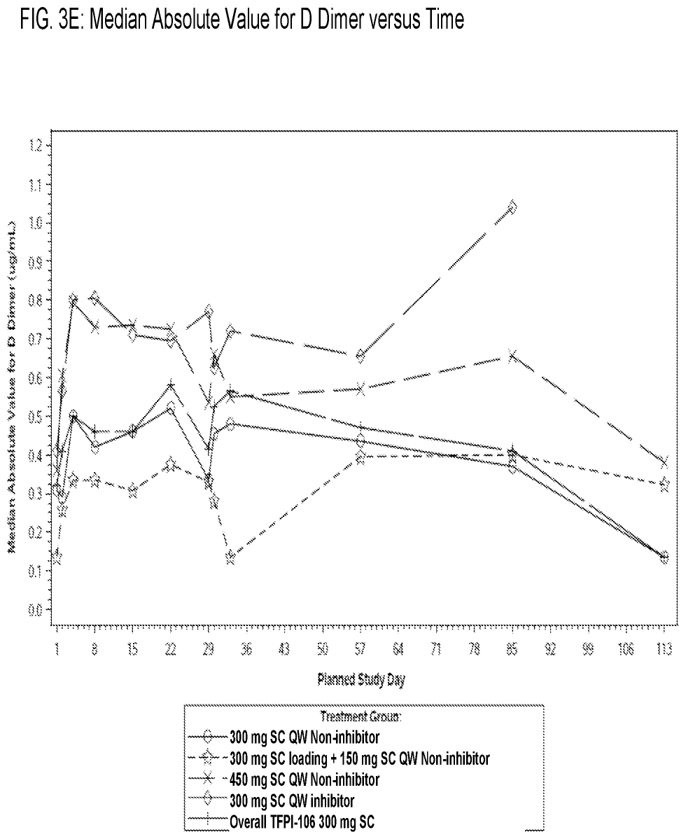

The instant invention relates to the unexpected observation that TFPI antagonist antibodies (e.g., TFPI 106, also known as PF-06741086) with a lower binding affinity (K_{D}) as compared to other TFPI antagonist antibodies (e.g., concizumab) have more desirable clinical uses because of their lower internalization rates and longer half-lives. In particular, it was determined that in contrast to the daily or biweekly dosing required for TFPI antagonist antibodies with high binding affinity to TFPI (e.g., K_{D}>5×10⁻¹¹ M), weekly dosing of TFPI antagonist antibodies with lower binding affinity (e.g., K_{D} of from about 5×10⁻⁷M to about 5×10⁻¹¹ M) was sufficient to provide a surprisingly high rate of response (see e.g., Eichler H et al. (2017) Concizumab (Anti-TFPI) exposure-response modeling in patients with Hemophilia A. Blood, 130 (Suppl 1), 3672, and Hilden et al. (2012) Hemostatic effect of a monoclonal antibody mAb 2021 blocking the interaction between FXa and TFPI in a rabbit hemophilia model. Blood. 119. 5871-8); versus Tables 3-4 and FIG. 3 disclosed herein). Weekly administration of 150 mg (with a loading dose of 300 mg) (Cohort 2) or 300 mg (Cohort 1) of exemplary anti-TFPI antibody, TFPI-106, provided a surprising 95% or a 85% reduction in Annualized bleeding rate (ABR) as compared to the ABR observed in a historical on demand group (i.e., hemophilic subjects treated with a coagulation therapy as needed to treat sudden hemorrhages; also known as historical standard), and a surprising 90% and 82% reduction in ABR as compared to the ABR prior to treatment (Tables 3-4). In addition, TFPI-106 appears to be safe and well-tolerated in these subjects. Accordingly, in some aspects, the instant disclosure provides dosing regimens for TFPI antagonist antibodies that allow for administration of lower effective dosages and/or less frequent dosing of the therapeutic antibodies.

### 2. Definitions

An “antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term “antibody” encompasses not only intact polyclonal or monoclonal antibodies, but also any antigen binding fragment (i.e., “antigen-binding portion”) or single chain thereof, fusion proteins comprising an antibody, and any other modified configuration of the immunoglobulin molecule that comprises an antigen recognition site including, for example without limitation, scFv, single domain antibodies (e.g., shark and camelid antibodies), maxibodies, minibodies, intrabodies, diabodies, triabodies, tetrabodies, v-NAR and bis-scFv (see, e.g., Hollinger and Hudson, 2005, Nature Biotechnology 23(9): 1126-1136). An antibody includes an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant regions that correspond to the different classes of immunoglobulins are called alpha, delta, epsilon, gamma, and mu, respectively. The subunit structures and three-dimensional configurations of different classes of immunoglobulins are well known.

The term “antigen binding portion” or “antigen binding fragment” of an antibody, as used herein, refers to one or more fragments of an intact antibody that retain the ability to specifically bind to a given antigen (e.g., TFPI). Antigen binding functions of an antibody can be performed by fragments of an intact antibody. Examples of binding fragments encompassed within the term “antigen binding portion” of an antibody include Fab; Fab′; F(ab′)₂; an Fd fragment consisting of the VH and CH1 domains; an Fv fragment consisting of the VL and VH domains of a single arm of an antibody; a single domain antibody (dAb) fragment (Ward et al., 1989, Nature 341:544-546), and an isolated complementarity determining region (CDR).

The term “monoclonal antibody” (Mab) refers to an antibody that is derived from a single copy or clone, including e.g., any eukaryotic, prokaryotic, or phage clone, and not the method by which it is produced. Preferably, a monoclonal antibody of the invention exists in a homogeneous or substantially homogeneous population.

“Humanized” antibody refers to forms of non-human (e.g. murine) antibodies that are chimeric immunoglobulins, immunoglobulin chains, or fragments thereof (such as Fv, Fab, Fab′, F(ab′)₂ or other antigen-binding subsequences of antibodies) that contain minimal sequence derived from non-human immunoglobulin. Preferably, humanized antibodies are human immunoglobulins (recipient antibody) in which residues from a complementary determining region (CDR) of the recipient are replaced by residues from a CDR of a non-human species (donor antibody) such as mouse, rat, or rabbit having the desired specificity, affinity, and capacity.

As used herein, “human antibody” means an antibody having an amino acid sequence corresponding to that of an antibody that can be produced by a human and/or which has been made using any of the techniques for making human antibodies known to those skilled in the art or disclosed herein. This definition of a human antibody includes antibodies comprising at least one human heavy chain polypeptide or at least one human light chain polypeptide. One such example is an antibody comprising murine light chain and human heavy chain polypeptides. Human antibodies can be produced using various techniques known in the art. In one embodiment, the human antibody is selected from a phage library, where that phage library expresses human antibodies (Vaughan et al., 1996, Nature Biotechnology, 14:309-314; Sheets et al., 1998, Proc. Natl. Acad. Sci. (USA) 95:6157-6162; Hoogenboom and Winter, 1991, J. Mol. Biol., 227:381; Marks et al., 1991, J. Mol. Biol., 222:581). Human antibodies can also be made by immunization of animals into which human immunoglobulin loci have been transgenically introduced in place of the endogenous loci, e.g., mice in which the endogenous immunoglobulin genes have been partially or completely inactivated. This approach is described in U.S. Pat. Nos. 5,545,807; 5,545,806; 5,569,825; 5,625,126; 5,633,425; and 5,661,016. Alternatively, the human antibody may be prepared by immortalizing human B lymphocytes that produce an antibody directed against a target antigen (such B lymphocytes may be recovered from an individual or may have been immunized in vitro). See, e.g., Cole et al. Monoclonal Antibodies and Cancer Therapy, Alan R. Liss, p. 77, 1985; Boerner et al., 1991, J. Immunol., 147 (1):86-95; and U.S. Pat. No. 5,750,373.

A “variable region” of an antibody refers to the variable region of the antibody light chain or the variable region of the antibody heavy chain, either alone or in combination. As known in the art, the variable regions of the heavy and light chain each consist of four framework regions (FRs) connected by three complementarity determining regions (CDRs) also known as hypervariable regions, contribute to the formation of the antigen binding site of antibodies. If variants of a subject variable region are desired, particularly with substitution in amino acid residues outside of a CDR region (i.e., in the framework region), appropriate amino acid substitution, preferably, conservative amino acid substitution, can be identified by comparing the subject variable region to the variable regions of other antibodies which contain CDR1 and CDR2 sequences in the same canonical class as the subject variable region (Chothia and Lesk, J. Mol. Biol. 196(4): 901-917, 1987). When choosing FR to flank subject CDRs, e.g., when humanizing or optimizing an antibody, FRs from antibodies which contain CDR1 and CDR2 sequences in the same canonical class are preferred.

“Complementarity Determining Region (CDR)” of a variable domain are amino acid residues within the variable region that are identified in accordance with the definitions of the Kabat, Chothia, the accumulation of both Kabat and Chothia, AbM, contact, and/or conformational definitions or any method of CDR determination well known in the art. Antibody CDRs may be identified as the hypervariable regions originally defined by Kabat et al. See, e.g., Kabat et al., 1992, Sequences of Proteins of Immunological Interest, 5th ed., Public Health Service, NIH, Washington D.C. The positions of the CDRs may also be identified as the structural loop structures originally described by Chothia and others. See, e.g., Chothia et al., 1989, Nature 342:877-883. Other approaches to CDR identification include the “AbM definition,” which is a compromise between Kabat and Chothia and is derived using Oxford Molecular's AbM antibody modeling software (now ACCELRYS®), or the “contact definition” of CDRs based on observed antigen contacts, set forth in MacCallum et al., 1996, J. Mol. Biol., 262:732-745. In another approach, referred to herein as the “conformational definition” of CDRs, the positions of the CDRs may be identified as the residues that make enthalpic contributions to antigen binding. See, e.g., Makabe et al., 2008, Journal of Biological Chemistry, 283:1156-1166. Still other CDR boundary definitions may not strictly follow one of the above approaches, but will nonetheless overlap with at least a portion of the Kabat CDRs, although they may be shortened or lengthened in light of prediction or experimental findings that particular residues or groups of residues or even entire CDRs do not significantly impact antigen binding. As used herein, a CDR may refer to CDRs defined by any approach known in the art, including combinations of approaches. The methods used herein may utilize CDRs defined according to any of these approaches. For any given embodiment containing more than one CDR, the CDRs may be defined in accordance with any of Kabat, Chothia, extended, AbM, contact, and/or conformational definitions.

As known in the art a “constant region” of an antibody refers to the constant region of the antibody light chain or the constant region of the antibody heavy chain, either alone or in combination.

An “epitope” refers to the area or region of an antigen (Ag) to which an antibody specifically binds, e.g., an area or region comprising residues that interacts with the antibody (Ab). Epitopes can be linear or conformational. In a linear epitope, all of the points of interaction between the protein and the interacting molecule (such as an antibody) occur linearly along the primary amino acid sequence of the protein. A “nonlinear epitope” or “conformational epitope” comprises noncontiguous polypeptides (or amino acids) within the antigenic protein to which an antibody specific to the epitope binds. The term “epitope” as used herein, is defined as a portion of an antigen to which an antibody can specifically bind as determined by any method well known in the art, for example, by conventional immunoassays. Alternatively, during the discovery process, the generation and characterization of antibodies may elucidate information about desirable epitopes. From this information, it is then possible to competitively screen antibodies for binding to the same epitope. An approach to achieve this is to conduct competition and cross-competition studies to find antibodies that compete or cross-compete with one another for binding to TFPI. That is, the antibodies compete for binding to the antigen such that the antibodies compete for binding to the antigen-binding site of an anti-TFPI antibody of the disclosure.

An antibody that “preferentially binds” or “specifically binds” (used interchangeably herein) to an epitope is a term well understood in the art, and methods to determine such specific or preferential binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. An antibody “specifically binds” or “preferentially binds” to a target if it binds with greater affinity, avidity, more readily, and/or with greater duration than it binds to other substances. For example, an antibody that specifically or preferentially binds to a TFPI epitope is an antibody that binds this epitope with greater affinity, avidity, more readily, and/or with greater duration than it binds to other TFPI epitopes or non-TFPI epitopes. It is also understood by reading this definition that, for example, an antibody (or moiety or epitope) that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding. “Specific binding” or “preferential binding” includes a compound, e.g., a protein, a nucleic acid, an antibody, and the like, which recognizes and binds to a specific molecule, but does not substantially recognize or bind other molecules in a sample. For instance, an antibody or a peptide receptor which recognizes and binds to a cognate ligand or binding partner (e.g., an anti-TFPI antibody that binds TFPI) in a sample, but does not substantially recognize or bind other molecules in the sample, specifically binds to that cognate ligand or binding partner. Thus, under designated assay conditions, the specified binding moiety (e.g., an antibody or an antigen-binding portion thereof or a receptor or a ligand binding portion thereof) binds preferentially to a particular target molecule and does not bind in a significant amount to other components present in a test sample.

A variety of assay formats may be used to select an antibody or peptide that specifically binds a molecule of interest. For example, solid-phase ELISA immunoassay, immunoprecipitation, Biacore™ (GE Healthcare, Piscataway, N.J.), KinExA, fluorescence-activated cell sorting (FACS), Octet™ (ForteBio, Inc., Menlo Park, Calif.) and Western blot analysis are among many assays that may be used to identify an antibody that specifically reacts with an antigen or a receptor, or ligand binding portion thereof, that specifically binds with a cognate ligand or binding partner. Typically, a specific or selective reaction will be at least twice the background signal or noise, more typically more than 10 times background, even more typically, more than 50 times background, more typically, more than 100 times background, yet more typically, more than 500 times background, even more typically, more than 1000 times background, and even more typically, more than 10,000 times background. Also, an antibody is said to “specifically bind” an antigen when the equilibrium dissociation constant (K_{D}) is ≤7 nM.

The term “binding affinity” is herein used as a measure of the strength of a non-covalent interaction between two molecules, e.g., and antibody, or fragment thereof, and an antigen. The term “binding affinity” is used to describe monovalent interactions (intrinsic activity).

Binding affinity between two molecules, e.g. an antibody, or fragment thereof, and an antigen, through a monovalent interaction may be quantified by determination of the dissociation constant (K_{D}). In turn, K_{D} can be determined by measurement of the kinetics of complex formation and dissociation using, e.g., the surface plasmon resonance (SPR) method (Biacore). The rate constants corresponding to the association and the dissociation of a monovalent complex are referred to as the association rate constants kₐ (or kₒₙ) and dissociation rate constant k_{d} (or k_{off}), respectively. K_{D} is related to kₐ and k_{d} through the equation K_{D}=k_{d}/kₐ. The value of the dissociation constant can be determined directly by well-known methods, and can be computed even for complex mixtures by methods such as those, for example, set forth in Caceci et al. (1984, Byte 9: 340-362). For example, the K_{D} may be established using a double-filter nitrocellulose filter binding assay such as that disclosed by Wong & Lohman (1993, Proc. Natl. Acad. Sci. USA 90: 5428-5432). Other standard assays to evaluate the binding ability of ligands such as antibodies towards target antigens are known in the art, including for example, ELISAs, Western blots, RIAs, and flow cytometry analysis, and other assays exemplified elsewhere herein. The binding kinetics and binding affinity of the antibody also can be assessed by standard assays known in the art, such as Surface Plasmon Resonance (SPR), e.g. by using a Biacore™ system, or KinExA.

An antibody that specifically binds its target may bind its target with a high affinity, that is, exhibiting a low K_{D} as discussed above, and may bind to other, non-target molecules with a lower affinity. For example, the antibody may bind to non-target molecules with a K_{D} of 1×10⁻⁶M or more, more preferably 1×10⁻⁵ M or more, more preferably 1×10⁻⁴ M or more, more preferably 1×10⁻³ M or more, even more preferably 1×10⁻² M or more. An antibody of the invention is preferably capable of binding to its target with an affinity that is at least two-fold, 10-fold, 50-fold, 100-fold 200-fold, 500-fold, 1,000-fold or 10,000-fold or greater than its affinity for binding to another non-TFPI molecule.

As used herein, the term “Tissue Factor Pathway Inhibitor or TFPI” refers to any form of TFPI and variants thereof that retain at least part of the activity of TFPI. TFPI is a multi-valent Kunitz domain containing protease inhibitor. Exemplary sequences of human, mouse, cynomolgus monkey, rabbit, and rat TFPI are provided in Table 5. Human TFPI is an extracellular glycoprotein with two predominant forms, TFPI-alpha and TFPI-beta. TFPI alpha, which is a 276 amino acid glycosylated protein (MW 43 kD) is the largest form of TFPI and consists of three Kunitz like domains and a basic carboxy terminal region. Alternative splicing produces TFPI-beta, which contains Kunitz Domain 1 (K1) and Kunitz Domain 2 (K2), but contains an alternative C-terminal portion lacking Kunitz domain 3 (K3) and the basic region. TFPI-beta is anchored to cell membranes through post-translational modification with a glycosylphosphatidylinositol (GPI) anchor.

The primary targets of TFPI are the proteases Factor Xa (FXa) and Factor VIIa (FVIIa), which are key factors in the initiation stage of the coagulation cascade. Biochemical analysis has revealed that K2 is the inhibitor of FXa, while K1 inhibits FVIIa-Tissue Factor complex. The role of K3 is unclear as it does not seem to have direct protease inhibitory activity, but may serve as a recognition site for the co-factor Protein S. The C-terminal domain, unique to TFPI-alpha, may be involved in the recognition of prothrombinase on the platelet surface.

Kunitz domain 1 (K1) corresponds to amino acid residues 26-76 of SEQ ID NO: 2, and Kunitz domain 2 (K2) corresponds to residues 91 to 147 of SEQ ID NO: 2. The K1 and K2 domains from other TFPI homologs, isoforms, variants, or fragments can be identified by sequence alignment or structural alignment against SEQ ID NO: 2.

The TFPI of the instant disclosure includes any naturally occurring form of TFPI which may be derived from any suitable organism. For example, TFPI may be a mammalian TFPI, such as human, mouse, rat, non-human primate, bovine, ovine, canine, feline, or porcine TFPI. In certain embodiments, the TFPI is human TFPI. The TFPI may be a mature form of TFPI (i.e., a TFPI protein that has undergone post-translational processing within a suitable cell). Such a mature TFPI protein may, for example, be glycosylated.

The TFPI of the instant disclosure includes any functional fragments or variants derived from a naturally occurring TFPI. A functional fragment of TFPI can be any part or portion of TFPI that retains the activity of a TFPI, such as the ability to inhibit Factor Xa (FXa), to inhibit the activity of FVIIa-tissue factor complex, and/or to function as a negative regulator of coagulation or hemostasis. For example, a functional fragment may comprise a Kunitz domain, such as the K1 domain, K2 domain, or both K1 and K2 domains of TFPI.

A functional variant can comprise one or more mutations as compared to a naturally occurring TFPI, and still retain the activity of a naturally occurring TFPI, such as the ability to inhibit Factor Xa (FXa), or the ability to inhibit the activity of FVIIa-tissue factor complex. For example, a variant may have various degrees of sequence identity to SEQ ID NOs: 1, 2, 3, 4, 5, 6, or 7, such as at least 60%, 70%, 80%, 85%, 90%, 91%, 92%, 93%, 94%, 95%, 96%, 97%, 98% or 99% identical to the sequence recited in SEQ ID NO: 1, SEQ ID NO: 2, SEQ ID NO: 3, SEQ ID NO: 4, SEQ ID NO: 5, SEQ ID NO: 6, or SEQ ID NO: 7.

The TPFI fragments, variants, isoforms and homologs of the invention should maintain important epitope residues (such as Ile105, Arg107, and Leu131, if TFPI-23 and TFPI-106 antibodies are used) as described herein. In addition, the TFPI may comprise five or more, eight or more, ten or more, twelve or more or fifteen or more surface accessible residues of the K2 domain of TFPI. A surface accessible residue is a residue having more than 40% relative accessibility.

For example, for the K2 domain of TFPI (see, e.g., SEQ ID NO: 2), the following amino acid residues have a greater than 40% relative accessibility: 94-95, 98, 100-110, 118-121, 123-124, 131, 134, 138-142 and 144-145. The TFPI may comprise five or more, eight or more, ten or more, twelve or more or fifteen or more of these residues, such as a fragment of TFPI that includes five or more, eight or more, ten or more, twelve or more or fifteen or more of these residues.

Specific amino acid residue positions in TFPI are numbered according to SEQ ID NO: 2 (human TFPIα K1K2K3). However, the present invention is not limited to SEQ ID NO: 2. Corresponding residues from other TFPI homologs, isoforms, variants, or fragments can be identified according to sequence alignment or structural alignment that is known in the art. For example, alignments can be done by hand or by using well-known sequence alignment programs such as ClustalW2, or “BLAST 2 Sequences” using default parameters. For example, Arg107 of SEQ ID NO: 2 corresponds to Arg104 of Mouse TFPI K1K2 (SEQ ID NO: 4).

As used herein, a “TFPI antagonist antibody” (interchangeably termed “anti-TFPI antibody”) refers to an antibody that is able to bind to TFPI and inhibit TFPI biological activity and/or downstream pathway(s) mediated by TFPI signaling. A TFPI antagonist antibody encompasses antibodies that block, antagonize, suppress or reduce (including significantly) TFPI biological activity, including downstream pathways mediated by TFPI signaling, such as ligand binding and/or elicitation of a cellular response to TFPI. For purpose of the present invention, it will be explicitly understood that the term “TFPI antagonist antibody” encompasses all the previously identified terms, titles, and functional states and characteristics whereby the TFPI itself, a TFPI biological activity (including but not limited to its ability to mediate any aspect of blood coagulation), or the consequences of the biological activity, are substantially nullified, decreased, or neutralized in any meaningful degree. In some embodiment, a TFPI antagonist antibody binds TFPI and prevents TFPI binding to and/or inhibition of Tissue Factor (TF)/Factor VIIa complex. In other embodiments, a TFPI antibody binds TFPI and prevents TFPI binding to and/or inhibition of Factor Xa. Examples of TFPI antagonist antibodies are provided herein.

The term “compete”, as used herein with regard to an antibody, means that binding of a first antibody, or an antigen-binding portion thereof, to an antigen reduces the subsequent binding of the same antigen by a second antibody or an antigen-binding portion thereof. In general, the binding a first antibody creates steric hindrance, conformational change, or binding to a common epitope (or portion thereof), such that the binding of the second antibody to the same antigen is reduced. Standard competition assays may be used to determine whether two antibodies compete with each other. One suitable assay for antibody competition involves the use of the Biacore technology, which can measure the extent of interactions using surface plasmon resonance (SPR) technology, typically using a biosensor system (such as a BIACORE® system). For example, SPR can be used in an in vitro competitive binding inhibition assay to determine the ability of one antibody to inhibit the binding of a second antibody. Another assay for measuring antibody competition uses an ELISA-based approach. Furthermore, a high throughput process for “binning” antibodies based upon their competition is described in International Patent Application No. WO2003/48731. Competition is present if one antibody (or fragment) reduces the binding of another antibody (or fragment) to TFPI. For example, a sequential binding competition assay may be used, with different antibodies being added sequentially. The first antibody may be added to reach binding that is close to saturation. Then, the second antibody is added. If the binding of second antibody to TFPI is not detected, or is significantly reduced (e.g., at least about 10%, at least about 20%, at least about 30%, at least about 40%, at least about 50%, at least about 60%, at least about 70%, at least about 80%, or at least about 90% reduction) as compared to a parallel assay in the absence of the first antibody (which value can be set as 100%), the two antibodies are considered as competing with each other.

An anti-TFPI antibody of the disclosure may have the ability to compete or cross-compete with another antibody of the disclosure for binding to TFPI as described herein. For example, an antibody of the disclosure may compete or cross-compete with antibodies described herein for binding to TFPI, or to a suitable fragment or variant of TFPI that is bound by the antibodies disclosed herein.

That is, if a first anti-TFPI antibody competes with a second antibody for binding to TFPI, but it does not compete where the second antibody is first bound to TFPI, it is deemed to “compete” with the second antibody (also referred to as unidirectional competition). Where an antibody competes with another antibody regardless of which antibody is first bound to TFPI, then the antibody “cross-competes” for binding to TFPI with the other antibody. Such competing or cross-competing antibodies can be identified based on their ability to compete/cross-compete with a known antibody of the disclosure in standard binding assays. For example, SPR, e.g., by using a Biacore™ system, ELISA assays or flow cytometry may be used to demonstrate competition/cross-competition. Such competition/cross-competition may suggest that the two antibodies bind to identical, overlapping or similar epitopes.

An anti-TFPI antibody of the disclosure may therefore be identified by a method that comprises a binding assay which assesses whether or not a test antibody is able to compete/cross-compete with a reference antibody of the disclosure (e.g., TFPI-23, TFPI-106) for a binding site on the target molecule.

The term “treatment” includes prophylactic and/or therapeutic treatments. If it is administered prior to clinical manifestation of a condition, the treatment is considered prophylactic. Therapeutic treatment includes, e.g., ameliorating or reducing the severity of a disease, or shortening the length of the disease.

An “individual” or a “subject” is a mammal, more preferably, a human. Mammals also include, but are not limited to, farm animals, sport animals, pets, primates (e.g., monkeys), horses, dogs, cats, mice and rats.

As used herein, a “historical standard annualized bleeding rate (ABR)” is the ABR obtained from a “historical standard” (i.e., control) group (also, referred to as “historical on demand” group or “on demand” group). This group is constructed to serve as the comparator (i.e., control) for the analysis of clinical efficacy. In some embodiments, the group includes data prospectively collected from hemophilic subjects that have been treated with coagulation replacement therapy as needed (i.e., on demand) to treat sudden haemorrhages. In some embodiments, data (i.e., ABR) obtained from subjects who were treated on demand in one or more of the following clinical studies can be used to construct the historical standard group: ReFacto AF 308262-4432 (B1831004), BeneFIX (B1821010), and BeneFIX 3090A1-400 (B1821004).

Reference to “about” a value or parameter herein includes (and describes) embodiments that are directed to that value or parameter per se. For example, description referring to “about X” includes description of “X.” Numeric ranges are inclusive of the numbers defining the range. Generally speaking, the term “about” refers to the indicated value of the variable and to all values of the variable that are within the experimental error of the indicated value (e.g. within the 95% confidence interval for the mean) or within 10 percent of the indicated value, whichever is greater. Where the term “about” is used within the context of a time period (years, months, weeks, days etc.), the term “about” means that period of time plus or minus one amount of the next subordinate time period (e.g. about 1 year means 11-13 months; about 6 months means 6 months plus or minus 1 week; about 1 week means 6-8 days; etc.), or within 10 percent of the indicated value, whichever is greater.

Where aspects or embodiments of the invention are described in terms of a Markush group or other grouping of alternatives, the present invention encompasses not only the entire group listed as a whole, but each member of the group individually and all possible subgroups of the main group, but also the main group absent one or more of the group members. The present invention also envisages the explicit exclusion of one or more of any of the group members in the claimed invention.

Unless otherwise defined, all technical and scientific terms used herein have the same meaning as commonly understood by one of ordinary skill in the art to which this invention belongs. Exemplary methods and materials are described herein, although methods and materials similar or equivalent to those described herein can also be used in the practice or testing of the present invention. All publications and other references mentioned herein are incorporated by reference in their entirety. In case of conflict, the present specification, including definitions, will control. Although a number of documents are cited herein, this citation does not constitute an admission that any of these documents forms part of the common general knowledge in the art. Throughout this specification and claims, the word “comprise,” or variations such as “comprises” or “comprising” will be understood to imply the inclusion of a stated integer or group of integers but not the exclusion of any other integer or group of integers. Unless otherwise required by context, singular terms shall include pluralities and plural terms shall include the singular. The materials, methods, and examples are illustrative only and not intended to be limiting.

It is understood that wherever embodiments are described herein with the language “comprising,” otherwise analogous embodiments described in terms of “consisting of” and/or “consisting essentially of” are also provided.

### 3. TFPI Antibody Dosing

The present invention is based on the surprising finding that, TFPI antagonist antibodies with a lower binding affinity (K_{D}) (e.g., TFPI 106; K_{D} in the nM range) as compared to other TFPI antagonist antibodies with higher binding affinity (e.g., concizumab; K_{D} in the pM range) provide a surprisingly high rate of response with more desirable dosing regimens, particularly for treating chronic conditions (e.g., hemophilia) that require repeated injections. The present invention provides dosing regimens that allow for administration of lower effective dosages and/or less frequent dosing of the therapeutic antibodies.

Accordingly, in some aspects, the present invention provides a method of shortening bleeding time, a method of treating or preventing a deficiency in blood coagulation or a bleeding disorder, a method of treating or preventing hemophilia A, B or C, a method of treating or preventing von Willebrand Disease (vWD), and a method for reducing the activity of TFPI. These methods comprise administering to a subject in need thereof an initial dose of about 50 mg to about 500 mg of an antibody or antigen binding fragment thereof that specifically binds to an epitope in Kunitz Domain 2 (K2) of Tissue Factor Pathway Inhibitor (TFPI), wherein said epitope comprises residues Ile105, Arg107, and Leu131, according to the numbering of SEQ ID NO: 2.

In some embodiments, the initial dose is selected from the group consisting of about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, and about 500 mg. In some embodiments, the initial dose is selected from the group consisting of about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg and about 450 mg. In some embodiments, the initial dose is selected from the group consisting of about 150 mg, about 175 mg, about 200 mg, about 250 mg, about 300 mg, about 350 mg, about 400 mg and about 450 mg. In some embodiments, the initial dose is about 150 mg. In some embodiments, the initial dose is about 200 mg. In some embodiments, the initial dose is about 250 mg. In some embodiments, the initial dose is about 300 mg. In some embodiments, the initial dose is about 350 mg. In some embodiments, the initial dose is about 400 mg. In some embodiments, the initial dose is about 450 mg.

In some embodiments, the initial dose is selected from the group consisting of 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, and 500 mg. In some embodiments, the initial dose is 300 mg. In some embodiments, the initial dose is 150 mg.

In some embodiments, one or more subsequent doses of the antibody or antigen binding fragment thereof are administered to the subject. The one or more subsequent doses can be about the same, less than or more than the initial dose.

In some embodiments, the one or more subsequent dose is administered in an amount that is about the same as the initial dose. In some embodiments, either the initial dose or the subsequent dose or both the initial dose and the subsequent dose are selected from the group consisting of about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, and about 500 mg. In some embodiments, either the initial dose or the subsequent dose or both the initial dose and the subsequent dose are selected from the group consisting of about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg and about 450 mg. In some embodiments, either the initial dose or the subsequent dose or both the initial dose and the subsequent dose are selected from the group consisting of about 150 mg, about 175 mg, about 200 mg, about 250 mg, about 300 mg, about 350 mg, about 400 mg and about 450 mg. In some embodiments, either or both of the initial dose or subsequent dose is about 150 mg In some embodiments, either or both of the initial dose or subsequent dose is about 200 mg. In some embodiments, either or both of the initial dose or subsequent dose is about 250 mg. In some embodiments, either or both of the initial dose or subsequent dose is about 300 mg. In some embodiments, either or both of the initial dose or subsequent dose is about 350 mg. In some embodiments, either or both of the initial dose or subsequent dose is about 400 mg. In some embodiments, either or both of the initial dose or subsequent dose is about 450 mg. In some embodiments, both the initial and subsequent dose is 300 mg. In some embodiments, both the initial and subsequent dose is 150 mg.

In some embodiments, the one or more subsequent dose is administered in an amount that is less than the initial dose. In some embodiments, the one or more subsequent dose is about two-thirds the initial dose. In some embodiments, the one or more subsequent dose is about one-half the initial dose. In some embodiments, the one or more subsequent dose is about one-third the initial dose.

In some embodiments, the initial dose is about 450 mg, and the subsequent dose is about 300 mg. In some embodiments, the initial dose is about 300 mg, and the subsequent dose is about 200 mg. In some embodiments, the initial dose is about 150 mg, and the subsequent dose is about 100 mg. In some embodiments, the initial dose is about 400 mg, and the subsequent dose is about 200 mg. In some embodiments, the initial dose is about 300 mg, and the subsequent dose is about 150 mg. In some embodiments, the initial dose is 300 mg, and the subsequent dose is 150 mg. In some embodiments, the initial dose is about 200 mg, and the subsequent dose is about 100 mg. In some embodiments, the initial dose is about 150 mg, and the subsequent dose is about 75 mg. In some embodiments, the subsequent dose is about one-third the initial dose. In some embodiments, the initial dose is about 450 mg, and the subsequent dose is about 150 mg. In some embodiments, the initial dose is about 300 mg, and the subsequent dose is about 100 mg. In some embodiments, the initial dose is about 150 mg, and the subsequent dose is about 50 mg.

In some embodiments, the one or more subsequent dose is administered in an amount that is more than the initial dose. In some embodiments, the one or more subsequent dose is twice the initial dose. In some embodiments, the initial dose is about 75 mg, and the subsequent dose is about 150 mg. In some embodiments, the initial dose is about 100 mg, and the subsequent dose is about 200 mg. In some embodiments, the initial dose is about 125 mg, and the subsequent dose is about 250 mg. In some embodiments, the initial dose is about 150 mg, and the subsequent dose is about 300 mg. In some embodiments, the initial dose is about 200 mg, and the subsequent dose is about 400 mg. In some embodiments, the initial dose is about 225 mg, and the subsequent dose is about 450 mg. In some embodiments, the initial dose is about 150 mg, and the subsequent dose is increased to about 175 mg, about 200 mg, about 225 mg, about 250 mg, about 300 mg, about 325 mg, about 350 mg, about 375 mg, about 400 mg, about 425 mg, or about 450 mg. In some embodiments, the initial dose is about 300 mg and the subsequent dose is increased to about 325 mg, about 350 mg, about 375 mg, about 400 mg, about 425 mg, or about 450 mg.

### 4. TFPI Antibody Dosing Intervals

The present invention encompasses embodiments wherein the one or more subsequent doses of the TFPI antagonist antibodies are administered once daily, once every 3 days, once every 6 days, twice a week, once a week (weekly), once every 2 weeks, once every 3 weeks, once every 4 weeks, once every 5 weeks, once every 6 weeks, once every 7 weeks or once every 8 weeks after the initial dose. In some embodiments, the one or more subsequent doses are administered about 1 week, 2 weeks, 3 weeks, or 4 weeks after the initial dose. In some embodiments, the one or more subsequent doses are administered twice a week after the initial dose. In some embodiments, the one or more subsequent doses are administered about 1 week (i.e., once a week, weekly) after the initial dose. In some embodiments, the one or more subsequent doses are administered once every 2 weeks after the initial dose. The initial and one or more subsequent doses can be selected from any of the amounts disclosed herein.

In some embodiments, the dosing regimen comprises weekly administration of TFPI antagonist antibodies described herein, wherein either the initial dose or the subsequent dose or both the initial dose and the subsequent dose are selected from the group consisting of about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, and about 500 mg. In some embodiments, either the initial dose or the subsequent dose or both the initial dose and the subsequent dose are selected from the group consisting of about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg and about 450 mg. In some embodiments, either the initial dose or the subsequent dose or both the initial dose and the subsequent dose are selected from the group consisting of about 150 mg, about 175 mg, about 200 mg, about 250 mg, about 300 mg, about 350 mg, about 400 mg and about 450 mg.

In some embodiments, the dosing regimen comprises weekly administration of TFPI antagonist antibodies described herein, wherein the initial and subsequent dose is about 150 mg. In some embodiments, the initial and subsequent dose is about 200 mg. In some embodiments, the initial and subsequent dose is about 250 mg. In some embodiments, the initial and subsequent dose is about 300 mg. In some embodiments, the initial dose is about 300 mg and the subsequent dose is about 150 mg. In some embodiments, the initial and subsequent dose is 300 mg. In some embodiments, the initial dose is 300 mg and the subsequent dose is 150 mg. In some embodiments, the initial and subsequent dose is about 450 mg. In some embodiments, the initial dose is about 150 mg, and the subsequent dose is increased to about 175 mg, about 200 mg, about 225 mg, about 250 mg, about 300 mg, about 325 mg, about 350 mg, about 375 mg, about 400 mg, about 425 mg, or about 450 mg. In some embodiments, the initial dose is about 150 mg and the subsequent dose is increased to about 300 mg. In some embodiments, the initial dose is about 300 mg, and the subsequent dose is increased to about 325 mg, about 350 mg, about 375 mg, about 400 mg, about 425 mg, or about 450 mg. In some embodiments, the initial dose is about 300 mg, and the subsequent dose is increased to about 400 mg. In some embodiments, the initial dose is about 300 mg, and the subsequent dose is increased to about 425 mg. In some embodiments, the initial dose is about 300 mg, and the subsequent dose is increased to about 450 mg.

The TFPI antagonist antibody or antigen binding fragment thereof described herein can be administered to the subject via any suitable route. It should be apparent to a person skilled in the art that the examples described herein are not intended to be limiting but to be illustrative of the techniques available. Accordingly, in some embodiments, the TFPI antagonist antibody or antigen binding fragment thereof is administered to a subject in accord with known methods, such as intravenous administration, e.g., as a bolus or by continuous infusion over a period of time, by subcutaneous, intramuscular, intraperitoneal, intracerebrospinal, transdermal, intra-articular, sublingually, intrasynovial, via insufflation, intrathecal, oral, inhalation or topical routes. Administration can be systemic, e.g., intravenous administration, or localized. The antibody may be administered once, at least twice, or for at least the period of time until the condition is treated, palliated or cured. The antibody generally will be administered for as long as the condition is present.

### 5. TFPI Antagonist Antibodies

The present invention relates to TFPI antagonist antibodies (i.e., anti-TFPI antibody) in general and their use. Exemplary TFPI antagonist antibodies include but are not limited to those described in WO 2017/029583, WO 2010/017196, WO 2011/109452, WO 2014/144577, WO 2010/072687, WO 2012/001087, WO 2014/140240, and WO 2015/007880, each of which is herein incorporated by reference in its entirety.

In some embodiments, the TFPI antagonist antibody is selected from the group consisting of TFPI 106 (also known as PF-06741086), TFPI-23, TFPI-107, concizumab (also known as mAb-2021, hz4F36), 2A8 (see, for example, US20170073428) and 2A8-200.

In some aspects, the antibody or antigen binding fragment thereof specifically binds to an epitope in Kunitz Domain 2 (K2) of Tissue Factor Pathway Inhibitor (TFPI), wherein the epitope comprises residues Ile105, Arg107, and Leu131, according to the numbering of SEQ ID NO: 2. In some embodiments, the anti-TFPI antibody does not bind to Kunitz Domain 1 (K1) of TFPI. In some embodiments, the epitope comprises further comprises residues Cys106, Gly108, Cys130, Leu131, and Gly132, according to the numbering of SEQ ID NO: 2. In some embodiments, the epitope further comprises Asp102, Arg112, Tyr127, Gly129, Met134, and Glu138, according to the numbering of SEQ ID NO: 2. In some embodiments, the epitope does not comprise: E100, E101, P103, Y109, T111, Y113, F114, N116, Q118, Q121, C122, E123, R124, F125, K126, and L140, according to the numbering of SEQ ID NO: 2. In some embodiments, the epitope does not comprise: D31, D32, P34, C35, K36, E100, E101, P103, Y109, K126, and G128, according to the numbering of SEQ ID NO: 2.

In some embodiments, the antibody or antigen binding fragment thereof comprises a heavy chain variable region (VH) comprising:

(a) a VH complementarity determining region one (CDR-H1) comprising the amino acid sequence of SEQ ID NO: 13.

(b) a VH complementarity determining region two (CDR-H2) comprising the amino acid sequence of SEQ ID NO: 14; and

(c) a VH complementarity determining region three (CDR-H3) comprising the amino acid sequence of SEQ ID NO: 15.

In some embodiments, the antibody or antigen binding fragment thereof comprises a VH comprising an amino acid sequence at least 90%, at least 95%, or at least 99% identical to an amino acid sequence selected from the group consisting of SEQ ID NOs: 16, 18, and 20. In some embodiments, the antibody or antigen binding fragment thereof comprises a VH comprising an amino acid sequence selected from the group consisting of SEQ ID NOs: 16, 18, and 20. In some embodiments, the antibody or antigen binding fragment thereof comprises a VH comprising the amino acid sequence of SEQ ID NO: 16. In some embodiments, the antibody or antigen binding fragment thereof comprises a VH comprising the amino acid sequence of SEQ ID NO: 18. In some embodiments, the antibody or antigen binding fragment thereof comprises a VH comprising the amino acid sequence of SEQ ID NO: 20.

In some embodiments, the antibody or antigen binding fragment thereof comprises a light chain variable region (VL) comprising:

(a) a VL complementarity determining region one (CDR-L1) comprising the amino acid sequence of SEQ ID NO: 8.

(b) a VL complementarity determining region two (CDR-L2) comprising the amino acid sequence of SEQ ID NO: 9; and

(c) a VL complementarity determining region three (CDR-L3) comprising the amino acid sequence of SEQ ID NO: 10.

In some embodiments, the antibody or antigen binding fragment thereof comprises a VL comprising an amino acid sequence at least 90%, at least 95%, or at least 99% identical to SEQ ID NO: 11. In some embodiments, the antibody or antigen binding fragment thereof comprises a VL comprising the amino acid sequence of SEQ ID NO: 11.

In some embodiments, the antibody or antigen binding fragment thereof comprises a heavy chain comprising the amino acid sequence of SEQ ID NO: 17. In some embodiments, the antibody or antigen binding fragment thereof comprises a heavy chain comprising the amino acid sequence of SEQ ID NO: 19. In some embodiments, the antibody or antigen binding fragment thereof comprises a heavy chain comprising the amino acid sequence of SEQ ID NO: 21. In some embodiments, the antibody or antigen binding fragment thereof comprises a light chain comprising the amino acid sequence of SEQ ID NO: 12.

In some embodiments, the antibody or antigen binding fragment thereof comprises:

(i) a heavy chain variable region (VH) comprising:

(a) a CDR-H1 comprising the amino acid sequence of SEQ ID NO: 13.

(b) a CDR-H2 comprising the amino acid sequence of SEQ ID NO: 14; and

(c) a CDR-H3 comprising the amino acid sequence of SEQ ID NO: 15, and

(ii) a light chain variable region (VL) comprising:

(a) a CDR-L1 comprising the amino acid sequence of SEQ ID NO: 8.

(b) a CDR-L2 comprising the amino acid sequence of SEQ ID NO: 9; and

(c) a CDR-L3 comprising the amino acid sequence of SEQ ID NO: 10.

In some embodiments, the antibody or antigen binding fragment thereof comprises a VH comprising the amino acid sequence of SEQ ID NO: 18, and a VL comprising the amino acid sequence of SEQ ID NO:11. In some embodiments, the antibody or antigen binding fragment thereof comprises a heavy chain comprising the amino acid sequence of SEQ ID NO: 19, and comprises a light chain comprising the amino acid sequence of SEQ ID NO: 12.

Exemplary antibodies of the present invention were deposited in the American Type Culture Collection, 10801 University Boulevard, Manassas, Va. 20110-2209, USA, on Jul. 22, 2015. Plasmid vector mAb-TFPI-106 VH having ATCC Accession No. PTA-122329 comprises a DNA insert encoding the heavy chain variable region of antibody TFPI-106, and plasmid vector mAb-TFPI-106 VL having ATCC Accession No. PTA-122328 comprises a DNA insert encoding the light chain variable region of antibody TFPI-106.

In some embodiments, the antibody or antigen binding fragment thereof comprises a VH comprising the amino acid sequence of SEQ ID NO: 16, and a VL comprising the amino acid sequence of SEQ ID NO:11. In some embodiments, the antibody or antigen binding fragment thereof comprises a heavy chain comprising the amino acid sequence of SEQ ID NO: 17, and comprises a light chain comprising the amino acid sequence of SEQ ID NO: 12.

In some embodiments, the antibody or antigen binding fragment thereof comprises a VH comprising the amino acid sequence of SEQ ID NO: 20, and a VL comprising the amino acid sequence of SEQ ID NO:11. In some embodiments, the antibody or antigen binding fragment thereof comprises a heavy chain comprising the amino acid sequence of SEQ ID NO: 21, and comprises a light chain comprising the amino acid sequence of SEQ ID NO: 12.

In some embodiments, the antibody or antigen binding fragment thereof comprises a heavy chain comprising the amino acid sequence of SEQ ID NO: 23, and comprises a light chain comprising the amino acid sequence of SEQ ID NO: 22. In some embodiments, the antibody or antigen binding fragment thereof comprises a heavy chain comprising the amino acid sequence of SEQ ID NO: 25, and comprises a light chain comprising the amino acid sequence of SEQ ID NO: 24.

In some embodiments, the antibody or antigen binding fragment thereof has a serum half-life of at least 25 hours, at least 29 hours, at least 30 hours at least 35 hours, at least 40 hours, at least 50 hours, at least 55 hours, at least 60 hours, at least 65 hours, at least 70 hours, at least 75 hours, at least 80 hours, at least 85 hours, at least 90 hours, at least 95 hours, at least 100 hours, at least 105 hours, at least 110 hours, at least 115 hours, at least 120 hours or at least 125 hours. In some embodiments, the antibody or antigen binding fragment thereof has a serum half-life of at least 25 hours, at least 29 hours, or at least 30 hours. In some embodiments, the antibody or antigen binding fragment thereof has a serum half-life of at least 29 hours. In some embodiments, the antibody or antigen binding fragment thereof has a serum half-life of at least 30 hours. In some embodiments, the antibody has a serum half-life of at least 115 hours, at least 120 hours or at least 125 hours.

In some embodiments, the antibody or antigen-binding fragment thereof has a binding affinity (K_{D}) of from about 5×10⁻⁷M to about 5×10⁻¹¹ M. In some embodiments, the antibody or antigen-binding fragment thereof has a K_{D} of from about 1×10⁻⁸M to about 1×10⁻¹° M (0.1 to 10 nm). In some embodiments, the antibody or antigen-binding fragment thereof has a K_{D}≤1 nM, ≤500 pM, ≤250 pM, ≤200 pM, ≤100 pM, ≤50 pM, ≤20 pM or ≤10 pM. In some embodiments, the antibody of antigen-binding fragment thereof does not have a K_{D} in the low pM range (i.e, ≤100 pM). In some aspects, the K_{D} is measured by surface plasmon resonance. In some aspects, surface plasmon resonance may be measured using a Biacore. In some aspects, the SPR may be measured using Biacore with captured antibody and solution phase human TFPI.

In some aspects, the antibody or antigen-binding fragment's subcutaneous bioavailability may be at least 10% relative to the intravenous bioavailability. In some aspects, the antibody or antigen-binding fragment's subcutaneous bioavailability may be at least 15% relative to the intravenous bioavailability. In some aspects, the antibody or antigen-binding fragment's subcutaneous bioavailability may be at least 20% relative to the intravenous bioavailability. In some aspects, the antibody or antigen-binding fragment's subcutaneous bioavailability may be at least 25% relative to the intravenous bioavailability. In some aspects, the antibody or antigen-binding fragment's subcutaneous bioavailability may be at least 27% relative to the intravenous bioavailability. In some aspects, the antibody or antigen-binding fragment's subcutaneous bioavailability may be at least 30% relative to the intravenous bioavailability. In some aspects, the antibody or antigen-binding fragment's subcutaneous bioavailability may be at least 35% relative to the intravenous bioavailability. In some aspects, the antibody or antigen-binding fragment's subcutaneous bioavailability may be at least 40% relative to the intravenous bioavailability. In some aspects, the antibody or antigen-binding fragment's subcutaneous bioavailability may be at least 50% relative to the intravenous bioavailability. In some aspects, the antibody or antigen-binding fragment's subcutaneous bioavailability may be at least 60% relative to the intravenous bioavailability. In some aspects, the antibody or antigen-binding fragment's subcutaneous bioavailability may be at least 70% relative to the intravenous bioavailability. In some aspects, the antibody or antigen-binding fragment's subcutaneous bioavailability may be at least 80% relative to the intravenous bioavailability. In some aspects, the antibody or antigen-binding fragment's subcutaneous bioavailability may be at least 90% relative to the intravenous bioavailability. In some aspects, the antibody or antigen-binding fragment's subcutaneous bioavailability may be at least 99% relative to the intravenous bioavailability.

In some aspects, the methods of the invention provide a markedly enhanced clinical benefit. The clinical benefit may be assessed by response rates and evaluation of disease progression. For example, clinical benefit may be assessed by measuring clotting time in a plasma based dilute prothrombin time (dPT) assay, by measuring clotting time in whole blood by thromboelastrography or rotational thromboelastometry, by measuring thrombin generation, by measuring FXa activity in the presence of TFPI, by measuring platelet accumulation in the presence of TFPI, by measuring fibrin generation in the presence of TFPI as measured by D-dimers, by measuring level of prothrombin fragment 1+2, or any combination thereof. The antibody or antigen binding fragments of the invention may have one or more of the following clinical benefits: (i) decreases clotting time as measured in a plasma based dilute prothrombin time (dPT) assay; (ii) reduces clotting time in whole blood as measured by thromboelastrography or rotational thromboelastometry; (iii) increases thrombin generation; (iv) increase FXa activity in the presence of TFPI; (v) enhances platelet accumulation in the presence of TFPI; (vi) increases fibrin generation in the presence of TFPI as measured by D-dimers; (vii) increases level of prothrombin fragment 1+2 or (viii) any combination thereof.

In some embodiments, the reduction in clotting time in whole blood is determined using whole blood obtained from a human subject having hemophilia A or B. In some embodiments, the reduction in clotting time in whole blood is determined using whole blood obtained from a human subject having (i) hemophilia A and inhibitory antibodies against human Factor VIII or (ii) hemophilia B and inhibitory antibodies against human Factor IX.

In some embodiments, the reduction in clotting time as measured in a dPT assay is determined using plasma obtained from a human subject having hemophilia A or B. In some embodiment, the reduction in clotting time as measured in a dPT assay is determined using plasma obtained from a human subject having (i) hemophilia A and inhibitory antibodies against human Factor VIII or (ii) hemophilia B and inhibitory antibodies against human Factor IX. In some embodiment, the increase in thrombin generation is determined using plasma obtained from a human subject having hemophilia A or B. In some embodiment, the increase in thrombin generation is determined using plasma obtained from a human subject having (i) hemophilia A and inhibitory antibodies against human Factor VIII or (ii) hemophilia B and inhibitory antibodies against human Factor IX.

In some embodiments, the methods of the invention provide clinical benefit as measured by a reduction of at least 20% in annualized bleeding rate (ABR) as compared to ABR observed in control subjects that have coagulation disorders. In some embodiments, the reduction in ABR is compared to control subjects that have hemophilia (e.g., hemophilia A or B). In some embodiments, the hemophilic control subjects have not been treated with coagulation replacement therapy. In some embodiments, the hemophilic control subjects have been treated with coagulation replacement therapy as needed to treat sudden haemorrhages (i.e., on demand). In some embodiments, a historical standard (i.e., on demand) group may be constructed using data prospectively collected during a hemophilia clinical study. The antibody or antigen-binding fragments of the invention may provide a reduction of at least 20% in mean ABR as compared to a historical On Demand group. The historical standard group may include data prospectively collected from hemophilia clinical studies, such as, but not limited to: ReFacto AF 308262-4432 (B1831004), BeneFIX (B1821010), and BeneFIX 3090A1-400 (B1821004). In some embodiments, the antibody or antigen-binding fragments of the invention provide a reduction of at least 25%, at least 30%, at least 35%, at least 40%, at least 45%, at least 50%, at least 55%, at least 60%, at least 65%, at least 70%, at least 75%, at least 80%, at least 82%, at least 85%, at least 90%, at least 95%, at least 96%, at least 98% or at least 99% in ABR as compared to a historical standard group. In some embodiments, the antibody or antigen-binding fragments of the invention provide a reduction of at least 75%, at least 80%, at least 85%, at least 90%, at least 95%, at least 96%, at least 98%, or at least 99% in ABR as compared to a historical standard group. In some embodiments, the antibody or antigen-binding fragments of the invention provide a reduction of at least 80% in ABR as compared to a historical standard group. In some embodiments, the antibody or antigen-binding fragments of the invention provide a reduction of at least 82% in ABR as compared to a historical standard group. In some embodiments, the antibody or antigen-binding fragments of the invention provide a reduction of at least 85% in ABR as compared to a historical standard group. In some embodiments, the antibody or antigen-binding fragments of the invention provide a reduction of at least 87% in ABR as compared to a historical standard group. In some embodiments, the antibody or antigen-binding fragments of the invention provide a reduction of at least 90% in ABR as compared to a historical standard group. In some embodiments, the antibody or antigen-binding fragments of the invention provide a reduction of at least 94% in ABR as compared to a historical standard group. In some embodiments, the antibody or antigen-binding fragments of the invention provide a reduction of at least 95% in ABR as compared to a historical standard group. In some embodiments, the antibody or antigen-binding fragments of the invention provide a reduction of at least 98% in ABR as compared to a historical standard group.

In some embodiments, the reduction in ABR is compared to that in the subject prior to treatment with the anti-TFPI antibody or antigen-binding fragment thereof. The antibody or antigen-binding fragments of the invention may provide a reduction of at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 98%, or at least 99% in ABR as compared to the ABR in the subject prior to treatment with the anti-TFPI antibody or antigen-binding fragment thereof.

In some embodiments, the mean ABR before administration of the TFPI antagonist antibody or antigen-binding fragment thereof, is at least 28 bleeds per year, at least 27.6 bleeds per year, at least 27 bleeds per year, at least 26 bleeds per year, at least 25 bleeds per year at least 24 bleeds per year, at least 23 bleeds per year, at least 22 bleeds per year, at least 22.6 bleeds per year, at least 21 bleeds per year, at least 20 bleeds per year, at least 19 bleeds per year, at least 18 bleeds per year, at least 17 bleeds per year, at least 16 bleeds per year, at least 15 bleeds per year, at least 14 bleeds per year, at least 13 bleeds per year, at least 12 bleeds per year, at least 11 bleeds per year, or at least 10 bleeds per year. In some embodiments, the mean ABR before administration of the TFPI antagonist antibody or antigen-binding fragment thereof, is at least 27 bleeds per year or at least 23 bleeds per year.

In some embodiments, the mean ABR after administration of the TFPI antagonist antibody or antigen-binding fragment thereof, is not more than 5 bleeds per year, not more than 4.2 bleeds per year, not more than 4 bleeds per year, not more than 3 bleeds per year, not more than 2 bleeds per year, not more than 1.5 bleeds per year, not more than 1 bleed per year, not more than 0.7 bleeds or no (i.e., 0) bleeds per year.

In some embodiments, administration of the antibody or antigen binding fragment is sufficient to achieve at least 1% of normal hemostatic activity. In some embodiments, administration of the antibody or antigen binding fragment is sufficient to achieve at least 2% of normal hemostatic activity. In some embodiments, administration of the antibody or antigen binding fragment is sufficient to achieve at least 3% of normal hemostatic activity. In some embodiments, administration of the antibody or antigen binding fragment is sufficient to achieve at least 4% of normal hemostatic activity. In some embodiments, administration of the antibody or antigen binding fragment is sufficient to achieve at least 5% of normal hemostatic activity. In some embodiments, administration of the antibody or antigen binding fragment is sufficient to achieve at least 10% of normal hemostatic activity. In some embodiments, administration of the antibody or antigen binding fragment is sufficient to achieve at least 15% of normal hemostatic activity. In some embodiments, administration of the antibody or antigen binding fragment is sufficient to achieve at least 20% of normal hemostatic activity. In some embodiments, administration of the antibody or antigen binding fragment is sufficient to achieve at least 25% of normal hemostatic activity. In some embodiments, administration of the antibody or antigen binding fragment is sufficient to achieve at least 30% of normal hemostatic activity. In some embodiments, administration of the antibody or antigen binding fragment is sufficient to achieve at least 40% of normal hemostatic activity. In some embodiments, administration of the antibody or antigen binding fragment is sufficient to achieve at least 50% of normal hemostatic activity. In some embodiments, administration of the antibody or antigen binding fragment is sufficient to achieve at least 55% of normal hemostatic activity. In some embodiments, administration of the antibody or antigen binding fragment is sufficient to achieve at least 60% of normal hemostatic activity. In some embodiments, administration of the antibody or antigen binding fragment is sufficient to achieve at least 65% of normal hemostatic activity. In some embodiments, administration of the antibody or antigen binding fragment is sufficient to achieve at least 70% of normal hemostatic activity. In some embodiments, administration of the antibody or antigen binding fragment is sufficient to achieve at least 75% of normal hemostatic activity. In some embodiments, administration of the antibody or antigen binding fragment is sufficient to achieve at least 80% of normal hemostatic activity. In some embodiments, administration of the antibody or antigen binding fragment is sufficient to achieve at least 85% of normal hemostatic activity. In some embodiments, administration of the antibody or antigen binding fragment is sufficient to achieve at least 90% of normal hemostatic activity. In some embodiments, administration of the antibody or antigen binding fragment is sufficient to achieve at least 95% of normal hemostatic activity. In some embodiments, administration of the antibody or antigen binding fragment is sufficient to achieve at least 99% of normal hemostatic activity.

Assays for measuring hemostatic activity are well known in the art (see e.g., WO 2017/029583, which is herein incorporated by reference in its entirety). Exemplary assays include, but are not limited to, rotational thromboelastography (ROTEM), thrombin generation assay (TGA), and dilute prothrombin time (dPT) assay. Normal hemostatic activity refers to an amount of hemostatic activity of untreated whole blood or plasma obtained from healthy (non-hemophilic) subjects.

### 6. Therapeutic Uses

Therapeutic methods are provided by the invention. A therapeutic method comprises administering a compound or composition of the invention to a subject in need thereof.

Exemplary therapeutic uses of the antibody and antibody fragments of the invention include shortening bleeding time in a subject in need thereof, treating or preventing deficiencies in blood coagulation or a blood disorder (e.g., hemophilia A, hemophilia B, hemophilia C, von Willebrand Disease (vWD), Factor VII deficiency, or Factor XI deficiency), treating or preventing thrombocytopenia, and treating or preventing platelet disorders (disorders of platelet function or number). The antibodies and antibody fragments may also be used for treating uncontrolled bleeding (for example, uncontrolled bleeding in indications such as trauma and hemorrhagic stroke). The antibodies and antibody fragments may also be used in prophylactic treatment (e.g., to treat or prevent bleeding before surgeries).

In particular, antibodies or antigen-binding fragments described herein can be used to treat deficiencies or defects in coagulation or disorders of coagulation. For example, the antibodies or antigen-binding fragments described herein may be used to reduce or inhibit the interaction of TFPI with FXa, or to reduce TFPI-dependent inhibition of the TF/FVIIa/FXa activity.

Accordingly, in some embodiments, the subject suffers from or is susceptible to a deficiency in blood coagulation or a blood disorder such as the following: In some embodiments, the subject suffers from or is susceptible to hemophilia A, B or C. In some embodiments, the subject suffers from or is susceptible to hemophilia A or B. In some embodiments, the subject suffers from or is susceptible to hemophilia A and has neutralizing antibodies (i.e., inhibitors) against coagulation factor VIII. In some embodiments, the subject suffers from or is susceptible to hemophilia B and has neutralizing antibodies (i.e., inhibitors) against coagulation factor IX. In some embodiments, the subject suffers from or is susceptible to von Willebrand Disease (vWD). In some embodiments, the subject suffers from or is susceptible to a platelet disorder. In some embodiments, the subject suffers from or is susceptible to a factor VII deficiency. In some embodiments, the subject suffers from or is susceptible to a factor XI deficiency.

TFPI antagonist antibodies or antigen-binding portions described herein may be used in combination with a clotting agent. The present invention provides for the separate, simultaneous or sequential administration of the antibodies of the invention with a clotting agent. Examples of clotting agent include, but are not limited to, factor VIIa, factor VIII, factor IX, tranexamic acid and bypass agents (e.g., anti-inhibitor coagulant complex or FEIBA).

### EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

### Example 1: Study Design

An open-label investigation of the safety, tolerability, PK, PD, and efficacy of multiple SC doses of TFPI-106 (i.e., PF-06741086) in males with severe hemophilia A or B (B7841002) was designed.

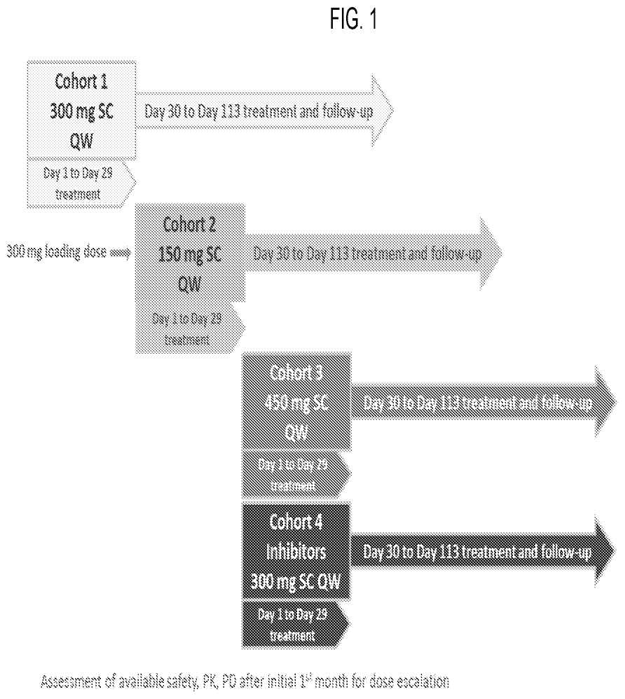

More specifically, multiple dose cohorts were enrolled and a dose-escalating study was carried out starting at a dose of 300 mg subcutaneous (SC). FIG. 1 shows a diagram illustrating the design of the study. Subjects were enrolled and assigned to treatment as follows:

Cohort 1: 7 subjects were enrolled and treated with 300 mg TFPI-106 SC once a week (QW) (n=7)

Cohort 2: 6 subjects were enrolled and treated with a loading dose of 300 mg SC TFPI-106, followed by 150 mg SC once a week (QW)

Cohort 3 (n=6): 6 subjects were enrolled and treated with 450 mg TFPI-106 SC once a week (QW)

Cohort 4: 7 subjects with inhibitors to Factor VIII or Factor IX were enrolled and treated with 300 mg TFPI-106 once a week (QW)

As noted above, subjects with inhibitors to FVIII or FIX were enrolled into a dedicated cohort (Cohort 4, treated with 300 mg SC QW). Additional subjects and/or cohorts are enrolled in the event that the number of dosing cohorts or size of dosing cohorts is increased. Increasing the number of dosing cohorts and/or size of the dosing cohorts may better define the dose range and/or clinical profile at the respective dose levels.

A historical standard (also referred to as historical On Demand or On Demand) group was constructed to serve as the comparator for the analysis of clinical efficacy. The On Demand group includes data prospectively collected from hemophilic subjects that have been treated with coagulation replacement therapy On Demand (i.e., as needed to treat sudden haemorrhages). In particular, data obtained from subjects who were treated On Demand in the following clinical studies were used to construct the historical On Demand group: ReFacto AF 308262-4432 (B1831004), BeneFIX (B1821010), and BeneFIX 3090A1-400 (B1821004). The resulting dataset was further filtered to match the key inclusion/exclusion criteria of the present study based on age and factor activity (18≤age 65 and factor activity ≤1%). All subjects meeting these criteria were included in the historical On Demand control group.

### Protocol Objectives and Endpoints

The primary objective of this study was to determine the safety and tolerability of multiple doses of TFPI-106 administered to severe hemophilia A and B subjects with and without inhibitors against FVIII or FIX.

The Primary Endpoints were as follows:

Frequency, severity and causal relationship of treatment emergent adverse events (TEAEs) and withdrawals due to TEAEs were assessed from Day 1 up to Day 113.

Frequency and magnitude of abnormal laboratory findings were assessed, including but not limited to, assays for hematology, prothrombin time/internalized normalized ratio (PT/INR), activated partial thromboplastin time (aPTT), urinalysis, anti-thrombin III activity and cardiac troponin I on Day 1 up to Day 113.

The subjects are also examined for changes from baseline in vital signs (e.g., blood pressure, pulse rate, temperature and respiration rate, electrocardiogram (ECG) and physical examination) from Day 1 up to Day 113.

Frequency, severity and casual relationship of infusion and injection site reactions are also assessed from Day 1 up to Day 113.

A Key Secondary Objective of the study is to assess the clinical efficacy of repeat dosing of TFPI-106. A Key Secondary Endpoint is to assess annualized rate of bleeding episodes from Day 1 up to Day 85.

### Example 2: Results of the Study

All subjects who receive at least 1 dose of TFPI-106 are included in the safety analyses and listings, i.e., the Safety Analysis Set (SAS).

The Per Protocol Analysis Set (PPAS) is a subset of the Safety Analysis Set. This set excludes subjects with major protocol deviations. Major protocol deviations include but are not limited to lack of compliance in study drug administration and violations on concomitant medications.

### Key Demographic and Baseline Characteristics of the Study Subjects

The key demographic and baseline characteristics of the population assigned to treatment are summarized in Table 1.

TABLE 1
Key Demographic and baseline characteristic (Safety Analysis Set)

| | | | 300 mg SC | | | | | | | |
|---|---|---|---|---|---|---|---|---|---|---|
| | 300 mg SC | | loading + 150 mg | | 450 mg SC | | 300 mg SC | | Overall - | |
| | QW Non-inhibitor | | QW Non-inhibitor | | QW Non-inhibitor | | QW inhibitor | | 300 mg SC QW | |
| All Males | (N = 7) | | (N = 6) | | (N = 6) | | (N = 5) | | (N = 12) | |
| Age (Years) | 7 | (100%) | 6 | (100%) | 6 | (100%) | 7 | (100%) | 14 | (100%) |
| Mean (SD) | 31.9 | (8.17) | 28.7 | (8.31) | 41.7 | (15.87) | 44.1 | (9.44) | 38.0 | (10.61) |
| Median | 28.0 | (26, 49) | 25.5 | (21, 43) | 44.5 | (19, 58) | 40 | (36, 63) | 36.5 | (26, 63) |
| (min, max) | | | | | | | | | | |
| Race | | | | | | | | | | |
| White | 3 | (42.9%) | 2 | (33.3%) | 6 | (100%) | 3 | (42.9%) | 6 | (42.9%) |
| Black or | 4 | (57.1%) | 4 | (66.7%) | 0 | | 4 | (57.1%) | 8 | (57.1%) |
| African | | | | | | | | | | |
| American | | | | | | | | | | |
| Ethnicity | | | | | | | | | | |
| Hispanic | 5 | (71.4%) | 2 | (33.3%) | 3 | (50%) | 1 | (14.3%) | 6 | (42.9%) |
| or Latino | | | | | | | | | | |
| Not Hispanic | 2 | (28.6%) | 4 | (66.7%) | 3 | (50%) | 6 | (85.7%) | 8 | (57.1%) |
| or Latino | | | | | | | | | | |
| Weight (kg) | | | | | | | | | | |
| n | 7 | | 6 | | 6 | | 7 | | 14 | |
| Mean (SD) | 75.89 | (13.245) | 62.28 | (9.832) | 83.95 | (7.149) | 72.71 | (16.401) | 74.30 | (14.416) |
| Median | 78.00 | (53.2, 92.0) | 61.60 | (50.2, 79.8) | 82.85 | (74.8, 94.8) | 67.00 | (52.0, 96.0) | 73.85 | (52.0, 96.0) |
| (min, max) | | | | | | | | | | |
| Body Mass | | | | | | | | | | |
| Index (BMI) | | | | | | | | | | |
| (kg/m²) | | | | | | | | | | |
| n | 7 | | 6 | | 6 | | 7 | | 14 | |
| Mean (SD) | 25.06 | (4.504) | 21.01 | (1.397) | 26.05 | (1.059) | 24.12 | (4.297) | 24.59 | (4.258) |
| Median | 25.28 | (18.0, 30.4) | 21.26 | (19.0, 22.8) | 26.30 | (24.7, 27.2) | 23.01 | (17.8, 29.6) | 24.42 | (17.8, 30.4) |
| (min, max) | | | | | | | | | | |
| Hemophilia A | 6 | (85.7%) | 5 | (83.3%) | 5 | (83.3%) | 7 | (100%) | 13 | (92.9%) |
| Hemophilia B | 1 | (14.3%) | 1 | (16.7%) | 1 | (16.7%) | 0 | | 1 | (7.1%) |
| Hemophilic | 7 | (100%) | 3 | (50%) | 6 | (100%) | 6 | (85.7%) | 13 | (92.9%) |
| Arthropathy | | | | | | | | | | |
| Target Joint | 7 | (100%) | 5 | (83.3%) | 6 | (100%) | 6 | (85.7%) | 13 | (92.9%) |
| (≥1) | | | | | | | | | | |

### Safety

To date, no clear or emerging safety signals have been identified for TFPI-106 at the three dose levels studied in hemophilia A and B subjects without inhibitors, or within data for hemophilia A or B subjects with inhibitors.

Conclusion: Adverse event, laboratory, coagulation assay, vital signs and ECG data indicate that the 300 mg SC QW and 150 mg SC QW regimens were generally safe and well-tolerated.

### Efficacy

The bleeding episodes that occurred during the study period, i.e., Day 1 to Day 85 are listed below (Table 2). One subject in Cohort 3 was dose modified down to 300 mg SC QW (from 450 mg SC) at Day 30 due to multiple severe injection site reactions. This subject had two spontaneous bleeding episodes after the dose modification.

TABLE 2
Listing of Bleeding Events in the PPAS

| | | Study Day of | | BLEEDING | Target Joint |
|---|---|---|---|---|---|
| Cohort | Patient | Bleeding | BLEED TYPE | SITE(S) | Bleed |
| 1 | 10021005 | 31 | TRAUMATIC | LEFT ELBOW | N |
| 1 | 10021007 | 34 | SPONTANEOUS | LEFT KNEE | Y |
| 1 | 10021007 | 84 | TRAUMATIC | RIGHT ANKLE | N |
| 1 | 10021009 | 12 | SPONTANEOUS | ELBOW LEFT | Y |
| 1 | 10021009 | 45 | SPONTANEOUS | RIGHT ELBOW | Y |
| 1 | 10041002 | 2 | SPONTANEOUS | LEFT ELBOW | N |
| 2 | 10021013 | 37 | TRAUMATIC | RIGHT ARM | N |
| 2 | 10031004 | 66 | SPONTANEOUS | LEFT ELBOW | Y |
| 3 | 10021015* | 13 | TRAUMATIC | LEFT KNEE | Y |
| 3 | 10021015* | 70 | SPONTANEOUS | LEFT ELBOW | N |
| 3 | 10021015* | 77 | SPONTANEOUS | LEFT ELBOW | N |
| 3 | 10041005 | 11 | SPONTANEOUS | RIGHT KNEE | N |
| 3 | 10041005 | 51 | SPONTANEOUS | RIGHT KNEE | N |
| 3 | 10041005 | 78 | SPONTANEOUS | INTRACRANIAL** | N |
| 4 | 10031008 | 74 | SPONTANEOUS | RECTAL | N |

*Subject 10021015 switched from 450 mg SC QW to 300 mg SC QW on Day 30 due to multiple severe injection site reactions.
**Unconfirmed intracranial bleed. No confirmatory investigations (e.g., CT scan) performed.

The key secondary endpoint of the study is annualized bleeding rate (ABR) from Day 1 to Day 85. The ABR from cohorts 1 to 3 in the PPAS was compared to the historical On Demand group using a negative binomial model (Table 3). The model based ABR was 4.2 for Cohort 1 (300 mg SC QW), 1.5 for Cohort 2 (300 mg SC loading dose followed by 150 mg SC QW), 4.2 for Cohort 3 (450 mg SC QW), 2.5 for Cohort 4 (300 mg SC QW inhibitors) and 27.6 for the historical On Demand group respectively. The percent reduction in ABR compared to the historical On Demand group was 85% (80% Cl: 71%, 92%; p=0.0002) for Cohort 1 (300 mg SC QW), 95% (80% Cl: 86%, 98%; p=0.0001) for Cohort 2 (300 mg SC loading dose followed by 150 mg SC QW), 85% (80% Cl 71%, 92%; p=0.0002) for Cohort 3 (450 SC QW) and 98% (80% Cl 90%, 99%; p=0.0005) for Cohort 4 (300 mg SC QW inhibitors) (Table 3). The median ABR was 4.2 for Cohort 1 (300 mg SC QW), 0 for Cohort 2 (300 mg SC loading dose followed by 150 mg SC QW), 0 for Cohort 3 (450 SC QW), 0 for Cohort 4 (300 mg SC QW inhibitors) and 22.6 for the historical On Demand group respectively.

The percent reduction in ABR compared to the pre-treatment period was 82% (80% Cl: 69%, 89%; p<0.0001) for Cohort 1 (300 mg SC QW); 90% (80% Cl: 78%, 96%; p=0.0002) for Cohort 2 (300 mg SC loading dose followed by 150 mg SC QW), 80% (80% Cl: 53%, 91%; p=0.0154) for Cohort 3 (450 SC QW) and 96% (80% Cl 86%, 99%; p=0.0005) for Cohort 4 (300 mg SC QW inhibitors) (Table 4).

TABLE 3
Annualized Bleeding Rate versus Historical On Demand Group (PPAS)

| | | | | | | 300 mg | | |
|---|---|---|---|---|---|---|---|---|
| | Historical | | | | | loading + | | |
| | On | | 300 mg | | | 150 mg | | |
| | Demand | | SC QW | | p-value | SC QW | | p-value |
| N | 65 | | 6 | | | 6 | | |
| ABR | 27.6 | (24.7, 30.9) | 4.2 | (2.2, 7.9) | | 1.5 | (0.6, 3.9) | |
| (80% CI)* | | | | | | | | |
| Ratio of | | | 0.15 | (0.08, 0.29) | | 0.05 | (0.02, 0.14) | |
| ABR | | | | | | | | |
| (80% CI) | | | | | | | | |
| % Reduction | | | 85% | (71%, 92%) | 0.0002* | 95% | (86%, 98%) | 0.0001* |
| (80% CI) | | | | | | | | |
| Median | 22.6 | (0.5, 67.6) | 4.2 | (0, 8.5) | <0.0001# | 0 | (0, 5.5) | <0.0001# |
| ABR | | | | | | | | |
| (Range) | | | | | | | | |

| | | | | 300 mg | | |
|---|---|---|---|---|---|---|
| | 450 mg | | | SC QW | | |
| | SC QW | | p-value | Inhibitor | | p-value |
| N | 6 | | | 6 | | |
| ABR | 4.2 | (2.2, 7.9) | | 0.72 | (0.19, 2.73) | |
| (80% CI)* | | | | | | |
| Ratio of | 0.15 | (0.08, 0.29) | | 0.03 | (0.01, 0.10) | |
| ABR | | | | | | |
| (80% CI) | | | | | | |
| % Reduction | 85% | (71%, 92%) | 0.0002* | 98% | (90%, 99%) | 0.0005* |
| (80% CI) | | | | | | |
| Median | 0 | (0, 12.6) | <0.0001# | 0 | (0, 3.9) | <0.0001# |
| ABR | | | | | | |
| (Range) | | | | | | |

*Negative binomial mode;
#Exact Wilcoxon Rank Sum test

TABLE 4
Annualized Bleeding Rate versus Pre-treatment Look Back in PPAS

| | | | | 300 mg | | | | | | | | |
|---|---|---|---|---|---|---|---|---|---|---|---|---|
| | | | | loading + | | | | | | 300 mg | | |
| | 300 mg | | | 150 mg | | | 450 mg | | | SC QW | | |
| | SC QW | | p-value | SC QW | | p-value | SC QW | | p-value | Inhibitor | | p-value |
| N | 6 | | | 6 | | | 6 | | | 6 | | |
| Pre- | 23 | (19.7, 26.9) | | 14.7 | (13.9, 15.5) | | 20.3 | (15.8, 26.2) | | 17.3 | (16.0, 18.8) | |
| Treatment | | | | | | | | | | | | |
| Look Back- | | | | | | | | | | | | |
| Mean ABR | | | | | | | | | | | | |
| ABR (80% CI)* | 4.2 | (2.7, 6.5) | | 1.5 | (0.7, 3.2) | | 4.2 | (2.0, 8.7) | | 0.7 | (0.2, 2.3) | |
| Ratio of ABR | 0.18 | (0.11, 0.31) | | 0.10 | (0.04, 0.22) | | 0.20 | (0.09, 0.47) | | 0.04 | (0.01, 0.14) | |
| (80% CI) | | | | | | | | | | | | |
| % Reduction | 82% | (69%, 89%) | <0.0001* | 90% | (78%, 96%) | 0.0002* | 80% | (53%, 91%) | 0.0154* | 96% | (86%, 99%) | 0.0005 |
| (80% CI) | | | | | | | | | | | | |
| Pre- | 24 | (12, 30) | | 15 | (12, 16) | | 17 | (12, 42) | | 18 | (12, 20) | |
| Treatment | | | | | | | | | | | | |
| Look Back- | | | | | | | | | | | | |
| Median ABR | | | | | | | | | | | | |
| Median ABR | 4.2 | (0, 8.5) | 0.0313# | 0 | (0, 5.5) | 0.0313# | 0 | (0, 12.6) | 0.0313# | 0 | (0, 3.9) | 0.0313# |
| (Range) | | | | | | | | | | | | |

*Negative binomial model;
#Wilcoxon Signed Rank test

### Immunogenicity

There were no confirmed anti-drug antibody (ADA) positive subjects in Cohort 1 (300 mg SC QW) or 2 (150 mg SC QW). Other cohorts are being assessed.

### Pharmacokinetics (PK)

A population PK model of TFPI-106 was established based on data from the previous first-in-human (FIH) study (B7841001) in healthy subjects.

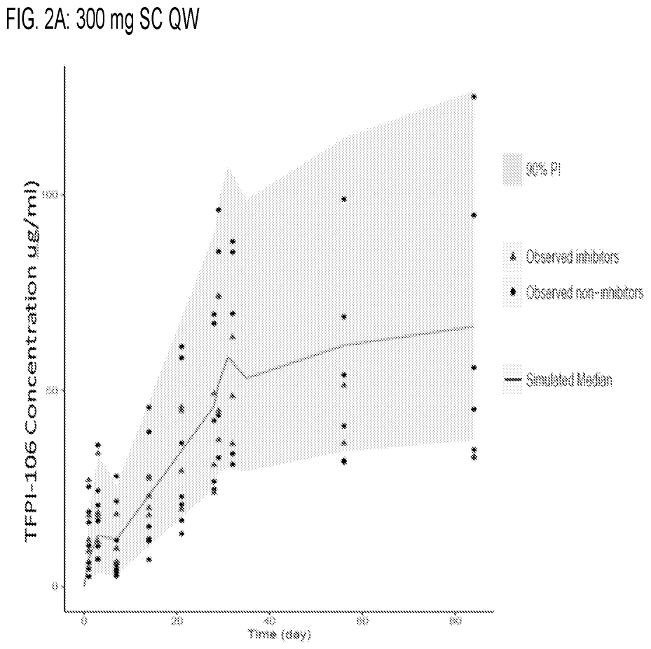

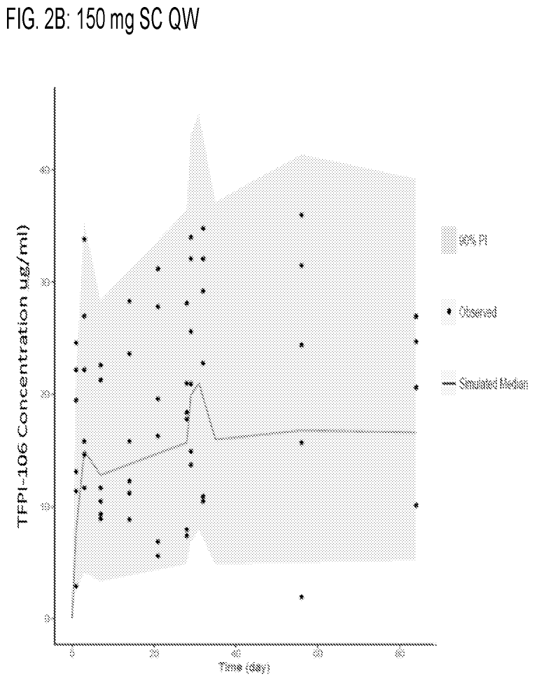

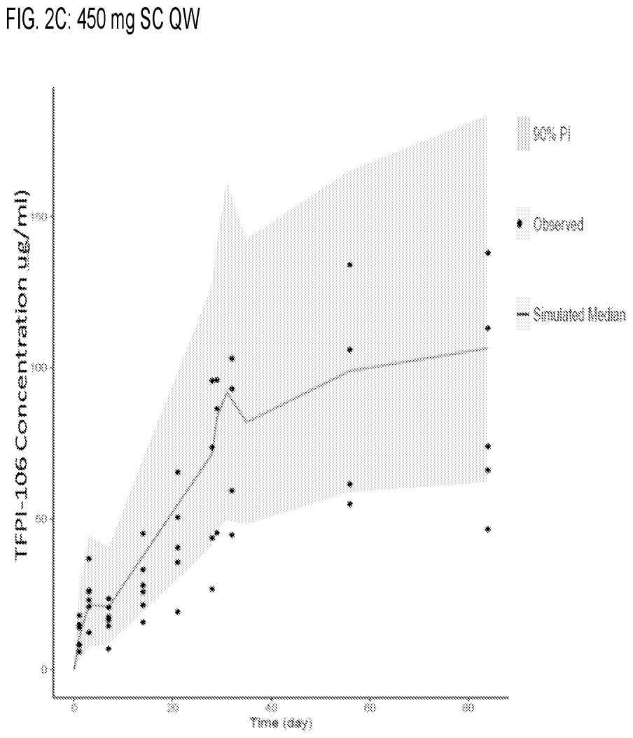

Distribution of key demographic information of subjects in the present study (B7841002) was used to simulate TFPI-106 exposure. The observed plasma concentrations from the present study (B7841002) were overlaid with model predictions for Cohort 1 (300 mg SC QW) (FIG. 2A), for Cohort 2 (300 mg SC loading dose followed by 150 mg SC QW) (FIG. 2B) and for Cohort 3 (450 mg SC QW) (FIG. 2C). The observed TFPI-106 exposures from Cohort 1 and 2 are consistent with model predictions. These data indicate that there was no apparent difference in PK between healthy subjects in the FIH study and hemophilia patients in the present study.

### Pharmacodynamics (PD)

The PD endpoints evaluated reflected target binding by TFPI-106 (total TFPI levels) or downstream pharmacologic effects of TFPI inhibition (i.e., thrombin generation assay (TGA), prothrombin fragment 1+2 (PF1+2) formation, dilute prothrombin time (dPT) and D-dimer formation).

Median values (total TFPI, TGA peak thrombin and D Dimer) or median change from baseline (dPT & PF1+2) vs. time by dose cohorts are shown in FIG. 3. Treatment related changes were observed for all available PD endpoints. These included (i) dose-dependent increases in total TFPI that are consistent with binding of TFPI with TFPI-106 (FIG. 3A); (ii) normalization (i.e., 90% CI [44, 176 nM] derived using log transformation of pre-dose peak thrombin values from all subjects in the FIH study (B7841001) to represent the normal range in healthy subjects) of TGA peak thrombin (FIG. 3B); (iii) dose-dependent increase in PF1+2 (FIG. 3C); (iv) shortening of dilute prothrombin time (dPT) (FIG. 3D); and (v) increase in D-dimer (FIG. 3E). These results indicate that treatment with TFPI-106 increased coagulation activity and normalization of thrombin generation.

FVIII and FIX deficiency (≤1% activity in severe hemophilia A and B, respectively) results in insufficient factor X (FX) activation, reduced thrombin generation and the subsequent inability to cleave sufficient levels of fibrinogen to generate fibrin for clot formation. Median pre-treatment peak thrombin for hemophilia subjects in Cohort 1 (22.1 nM, range 16.2-60.0) and Cohort 2 (26.8 nM, range 14.3-57.2) are indicative of this reduced thrombin generation. As a comparison, healthy volunteers (with normal FVIII and FIX levels) from the Phase 1 FIH study (B7841001) had pre-treatment thrombin generation approximately 4-fold higher (93.2 nm, range 71.4-132.7). Following treatment with TFPI-106, all subjects in Cohort 1, Cohort 2 and Cohort 3, at steady state concentrations, achieved peak thrombin generation approximating the normal range exhibited by healthy volunteers from the FIH study (B7841001).

TABLE 5
Sequences

| Seq ID | Description | Sequence |
|---|---|---|
| 1 | Human TFPIα K1K2 | DSEEDEEHTI ITDTELPPLK LMHSFCAFKA DDGPCKAIMK |
| | (Accession #P10646, | RFFFNIFTRQ CEEFIYGGCE GNQNRFESLE ECKKMCTRDN |
| | residues 29-177) | ANRIIKTTLQ QEKPDFCFLE EDPGICRGYI TRYFYNNQTK |
| | | QCERFKYGGC LGNMNNFETL EECKNICED |
| 2 | Human TFPIα | DSEEDEEHTI ITDTELPPLK LMHSFCAFKA DDGPCKAIMK |
| | K1K2K3 Accession | RFFFNIFTRQ CEEFIYGGCE GNQNRFESLE ECKKMCTRDN |
| | #P10646, residues | ANRIIKTTLQ QEKPDFCFLE EDPGICRGYI TRYFYNNQTK |
| | 29-282) | QCERFKYGGC LGNMNNFETL EECKNICEDG PNGFQVDNYG |
| | | TQLNAVNNSL TPQSTKVPSL FEFHGPSWCL TPADRGLCRA |
| | | NENRFYYNSV IGKCRPFKYS GCGGNENNFT SKQECLRACK |
| | | KGFIQRISKG GLIK |
| 3 | Human TFPI2 | DAAQEPTGNN AEICLLPLDY GPCRALLLRY YYDRYTQSCR |
| | K1K2K3 Accession # | QFLYGGCEGN ANNFYTWEAC DDACWRIEKV PKVCRLQVSV |
| | P48307.1, residues | DDQCEGSTEK YFFNLSSMTC EKFFSGGCHR NRIENRFPDE |
| | 23-211) | ATCMGFCAPK KIPSFCYSPK DEGLCSANVT RYYFNPRYRT |
| | | CDAFTYTGCG GNDNNFVSRE DCKRACAKA |
| 4 | Mouse TFPI K1K2 | LSEEADDTDS ELGSMKPLHT FCAMKADDGP CKAMIRSYFF |
| | (Accession #O54819, | NMYTHQCEEF IYGGCEGNEN RFDTLEECKK TCIPGYEKTA |
| | residues 29-174) | VKAASGAERP DFCFLEEDPG LCRGYMKRYL YNNQTKQCER |
| | | FVYGGCLGNR NNFETLDECK KICENP |
| 5 | Cynomolgus Monkey | DSEEDEEYTI ITDTELPPLK LMHSFCAFKP DDGPCKAIMK |
| | TFPI K1K2 | RFFFNIFTRQ CEEFIYGGCG GNQNRFESME ECKKVCTRDN |
| | (Accession #Q2PFV4, | VNRIIQTALQ KEKPDFCFLE EDPGICRGYI TRYFYNNQSK |
| | residues 29-177) | QCERFKYGGC LGNMNNFETL EECKNTCED |
| 6 | Rabbit TFPI K1K2 | AAEEDEEFTN ITDIKPPLQK PTHSFCAMKV DDGPCRAYIK |
| | (Accession #P19761, | RFFFNILTHQ CEEFIYGGCE GNENRFESLE ECKEKCARDY |
| | residues 29-173) | PKMTTKLTFQ KGKPDFCFLE EDPGICRGYI TRYFYNNQSK |
| | | QCERFKYGGC LGNLNNFESL EECKNTCEN |
| 7 | Rat TFPI K1K2 | LPEEDDDTIN TDSELRPMKP LHTFCAMKAE DGPCKAMIRS |
| | (Accession #Q02445, | YYFNMNSHQC EEFIYGGCRG NKNRFDTLEE CRKTCIPGYK |
| | residues 29-176) | KTTIKTTSGA EKPDFCFLEE DPGICRGFMT RYFYNNQSKQ |
| | | CEQFKYGGCL GNSNNFETLE ECRNTCED |
| 8 | mAb-TFPI-23 LC | TGSSSNIGAG YDVH |
| | CDR1 | |
| 9 | mAb-TFPI-23 LC | GNSNRPS |
| | CDR2 | |
| 10 | mAb-TFPI-23 LC | QSYDSSLSGS GV |
| | CDR3 | |
| 11 | mAb-TFPI-23 VL | QSVLTQPPSV SGAPGQRVTI SCTGSSSNIG AGYDVHWYQQ |
| | CDR1, CDR2, CDR3 | LPGTAPKLLI YGNSNRPSGV PDRFSGSKSG TSASLAITGL |
| | are underlined | QAEDEADYYC QSYDSSLSGS GVFGGGTKLT VLG |
| 12 | mAb-TFPI-23 LC | QSVLTQPPSV SGAPGQRVTI SCTGSSSNIG AGYDVHWYQQ |
| | mAb-TFPI-106 LC | LPGTAPKLLI YGNSNRPSGV PDRFSGSKSG TSASLAITGL |
| | mAb-TFPI-107 LC | QAEDEADYYC QSYDSSLSGS GVFGGGTKLT VLGQPKAAPS |
| | CDR1, 2, 3 are | VTLFPPSSEE LQANKATLVC LISDFYPGAV TVAWKADSSP |
| | underlined. Variable | VKAGVETTTP SKQSNNKYAA SSYLSLTPEQ WKSHRSYSCQ |
| | sequence in italics | VTHEGSTVEK TVAPTECS |
| 13 | mAb-TFPI-23 HC | GFTFSSYAMS |
| | CDR1 | |
| 14 | mAb-TFPI-23 HC | AISGSGGSTY YADSVKG |
| | CDR2 | |
| 15 | mAb-TFPI-23 HC | LGATSLSAFD I |
| | CDR3 | |
| 16 | mAb-TFPI-23 VH | QVQLVESGGG LVQPGGSLRL SCAASGFTFS SYAMSWVRQA |
| | | PGKGLEWVSA ISGSGGSTYY ADSVKGRFTI SRDNSKNTLY |
| | | LQMNSLRAED TAVYYCAILG ATSLSAFDIW GQGTMVTVSS |
| 17 | mAb-TFPI-23 HC | QVQLVESGGG LVQPGGSLRL SCAASGFTFS SYAMSWVRQA |
| | CDR1, CDR2 and | PGKGLEWVSA ISGSGGSTYY ADSVKGRFTI SRDNSKNTLY |
| | CDR3 underlined. | LQMNSLRAED TAVYYCAILG ATSLSAFDIW GQGTMVTVSS |
| | Variable sequence in | ASTKGPSVFP LAPSSKSTSG GTAALGCLVK DYFPEPVTVS |
| | italics. Effector | WNSGALTSGV HTFPAVLQSS GLYSLSSVVT VPSSSLGTQT |
| | function mutations | YICNVNHKPS NTKVDKKVEP KSCDKTHTCP PCPAPEAAGA |
| | in bold. | PSVFLFPPKP KDTLMISRTP EVTCVVVDVS HEDPEVKFNW |
| | | YVDGVEVHNA KTKPREEQYN STYRVVSVLT VLHQDWLNGK |
| | | EYKCKVSNKA LPAPIEKTIS KAKGQPREPQ VYTLPPSREE |
| | | MTKNQVSLTC LVKGFYPSDI AVEWESNGQP ENNYKTTPPV |
| | | LDSDGSFFLY SKLTVDKSRW QQGNVFSCSV MHEALHNHYT |
| | | QKSLSLSPG |
| 18 | mAb-TFPI-106 VH | EVQLLESGGG LVQPGGSLRL SCAASGFTFS SYAMSWVRQA |
| | Human Ig lambda CL | PGKGLEWVSA ISGSGGSTYY ADSVKGRFTI SRDNSKNTLY |
| | with Q1E, V5L | LQMNSLRAED TAVYYCAILG ATSLSAFDIW GQGTMVTVSS |
| | mutations in bold | |
| 19 | mAb-TFPI-106 HC | [symbol] VQL [symbol] ESGGG LVQPGGSLRL SCAASGFTFS SYAMSWVRQA |
| | CDR1, CDR2 and | PGKGLEWVSA ISGSGGSTYY ADSVKGRFTI SRDNSKNTLY |
| | CDR3 underlined. | LQMNSLRAED TAVYYCAILG ATSLSAFDIW GQGTMVTVSS |
| | Variable sequence in | ASTKGPSVFP LAPSSKSTSG GTAALGCLVK DYFPEPVTVS |
| | italics. Q1E, V5L | WNSGALTSGV HTFPAVLQSS GLYSLSSVVT VPSSSLGTQT |
| | mutations in bold. | YICNVNHKPS NTKVDKKVEP KSCDKTHTCP PCPAPEAAGA |
| | Effector function | PSVFLFPPKP KDTLMISRTP EVTCVVVDVS HEDPEVKFNW |
| | mutations in bold. | YVDGVEVHNA KTKPREEQYN STYRVVSVLT VLHQDWLNGK |
| | | EYKCKVSNKA LPAPIEKTIS KAKGQPREPQ VYTLPPSREE |
| | | MTKNQVSLTC LVKGFYPSDI AVEWESNGQP ENNYKTTPPV |
| | | LDSDGSFFLY SKLTVDKSRW QQGNVFSCSV MHEALHNHYT |
| | | QKSLSLSPG |
| 20 | mAb-TFPI-107 VH | EVQLLESGGG LVQPGGSLRL SCAASGFTFS SYAMSWVRQA |
| | Human Ig lambda CL | PGKGLEWVSA ISGSGGSTYY ADSVKGRFTI SRDNSKNTLY |
| | with Q1E, V5L, I94K | LQMNSLRAED TAVYYCAKLG ATSLSAFDIW GQGTMVTVSS |
| | mutations in bold | |
| 21 | mAb-TFPI-107 HC | [symbol] VQLLESGGG LVQPGGSLRL SCAASGFTFS SYAMSWVRQA |
| | CDR1, CDR2 and | PGKGLEWVSA ISGSGGSTYY ADSVKGRFTI SRDNSKNTLY |
| | CDR3 underlined. | LQMNSLRAED TAVYYCA [symbol] LG ATSLSAFDIW GQGTMVTVSS |
| | Variable sequence in | ASTKGPSVFP LAPSSKSTSG GTAALGCLVK DYFPEPVTVS |
| | italics. Q1E, V5L, | WNSGALTSGV HTFPAVLQSS GLYSLSSVVT VPSSSLGTQT |
| | I94K mutations in | YICNVNHKPS NTKVDKKVEP KSCDKTHTCP PCPAPEAAGA |
| | bold. | PSVFLFPPKP KDTLMISRTP EVTCVVVDVS HEDPEVKFNW |
| | Effector function | YVDGVEVHNA KTKPREEQYN STYRVVSVLT VLHQDWLNGK |
| | mutations in bold. | EYKCKVSNKA LPAPIEKTIS KAKGQPREPQ VYTLPPSREE |
| | | MTKNQVSLTC LVKGFYPSDI AVEWESNGQP ENNYKTTPPV |
| | | LDSDGSFFLY SKLTVDKSRW QQGNVFSCSV MHEALHNHYT |
| | | QKSLSLSPG |
| 22 | 2A8-200 LC | DIELTQPPSV SVAPGQTARI SCSGDNLRNY YAHWYQQKPG |
| | CDR1, CDR2, CDR3 | QAPVVVIFYD VNRPSGIPER FSGSNSGNTA TLTISGTQAE |
| | underlined | DEADYYCQSW WDGVPVFGGG TKLTVLGQPK AAPSVTLFPP |
| | Variable sequence in | SSEELQANKA TLVCLISDFY PGAVTVAWKA DSSPVKAGVE |
| | italics | TTTPSKQSNN KYAASSYLSL TPEQWKSHRS YSCQVTHEGS |
| | | TVEKTVAPTE CS |
| 23 | 2A8-200 HC | QVQLVESGGG LVQPGGSLRL SCAASGFTFR SYGMDWVRQA |
| | CDR1, CDR2, CDR3 | PGKGLEWVSS IRGSRSSTYY ADSVKGRFTI SRDNSKNTLY |
| | underlined | LQMNSLRAED TAVYYCARLY RYWFDYWGQG TLVTVSSAST |
| | Variable sequence in | GKPSVFPLAP SSKSTSGGTA ALGCLVKDYF PEPVTVSWNS |
| | italics | GALTSGVHTF PAVLQSSGLY SLSSVVTVPS SSLGTQTYIC |
| | | NVNHKPSNTK VDKKVEPKSC DKTHTCPPCP APEAAGAPSV |
| | | FLFPPKPKDT LMISRTPEVT CVVVDVSHED PEVKFNWYVD |
| | | GVEVHNAKTK PREEQYNSTY RVVSVLTVLH QDWLNGKEYK |
| | | CKVSNKALPA PIEKTISKAK GQPREPQVYT LPPSREEMTK |
| | | NQVSLTCLVK GFYPSDIAVE WESNGQPENN YKTTPPVLDS |
| | | DGSFFLYSKL TVDKSRWQQG NVFSCSVMHE ALHNHYTQKS |
| | | LSLSPG |
| 24 | hz4F36 LC | DIVMTQTPLS LSVTPGQPAS ISCKSSQSLL ESDGKTYLNW |
| | CDR1, CDR2, CDR3 | YLQKPGQSPQ LLIYLVSILD SGVPDRFSGS GSGTDFTLKI |
| | underlined | SRVEAEDVGV YYCLQATHFP QTFGGGTKVE IKRTVAAPSV |
| | Variable sequence in | FIFPPSDEQL SKGTASVVCL LNNFYPREAK VQWKVDNALQ |
| | italics | SGNSQESVTE QDSKDSTYSL SSTLTLSKAD YEKHKVYACE |
| | | VTHQGLSSPV TKSFNRGEC |
| 25 | hz4F36 HC | EVQLVESGGG LVKPGGSLRL SCAASGFTFS NYAMSWVRQT |
| | CDR1, CDR2, CDR3 | PEKRLEWVAT ISRSGSYSYF PDSVQGRFTI SRDNAKNSLY |
| | underlined | LQMNSLRAED TAVYYCARLG GYDEGDAMDS WGQGTTVTVS |
| | Variable sequence in | SASTKGPSVF PLAPCSRSTS ESTAALGCLV KDYFPEPVTV |
| | italics | SWNSGALTSG VHTFPAVLQS SGLYSLSSVV TVPSSSLGTK |
| | | TYTCNVDHKP SNTKVDKRVE SKYGPPCPPC PAPEFLGGPS |
| | | VFLFPPKPKD TLMISRTPEV TCVVVDVSQE DPEVQFNWYV |
| | | DGVEVHNAKT KPREEQFNST YRVVSVLTVL HQDWLNGKEY |
| | | KCKVSNKGLP SSIEKTISKA KGQPREPQVY TLPPSQEEMT |
| | | KNQVSLTCLV KGFYPSDIAV EWESNGQPEN NYKTTPPVLD |
| | | SDGSFFLYSR LTVDKSRWQE GNVFSCSVMH EALHNHYTQK |
| | | SLSLSLGK |

The various features and embodiments of the present invention, referred to in individual sections above apply, as appropriate, to other sections, mutatis mutandis. Consequently features specified in one section may be combined with features specified in other sections, as appropriate. All references cited herein, including patents, patent applications, papers, text books, and cited sequence Accession numbers, and the references cited therein are hereby incorporated by reference in their entirety. In the event that one or more of the incorporated literature and similar materials differs from or contradicts this application, including but not limited to defined terms, term usage, described techniques, or the like, this application controls.

1. A method of shortening bleeding time in a subject in need thereof, comprising administering to the subject an initial dose of about 50 mg to about 500 mg of an antibody, or antigen binding fragment thereof, that specifically binds to an epitope in Kunitz Domain 2 (K2) of Tissue Factor Pathway Inhibitor (TFPI).

2-3. (canceled)

4. The method as claimed in claim 1, further comprising administering to the subject one or more subsequent doses of the antibody or antigen binding fragment thereof.

5. (canceled)

6. The method as claimed in claim 1, wherein either or both of the initial dose or subsequent dose is selected from the group consisting of about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, and about 500 mg.

7. The method as claimed in claim 6, wherein either or both of the initial dose or subsequent dose is about 150 mg.

8. The method as claimed in claim 6, wherein either or both of the initial dose or subsequent dose is about 300 mg.

9. The method as claimed in claim 6, wherein either or both of the initial dose or subsequent dose is about 450 mg.

10. The method as claimed in claim 6, wherein the initial dose is about 300 mg, and the subsequent dose is about 150 mg.

11-13. (canceled)

14. The method as claimed in claim 1, wherein the subsequent dose is administered about 1 week after the initial dose.

15. The method as claimed in claim 1, wherein the antibody or antigen binding fragment thereof is administered subcutaneously.

16-17. (canceled)

18. The method as claimed in claim 1, wherein the antibody or antigen binding fragment thereof comprises:
(i) a heavy chain variable region (VH) comprising:
(a) a VH complementarity determining region one (CDR-H1) comprising the amino acid sequence of SEQ ID NO: 13;
(b) a VH complementarity determining region two (CDR-H2) comprising the amino acid sequence of SEQ ID NO: 14; and
(c) a VH complementarity determining region three (CDR-H3) comprising the amino acid sequence of SEQ ID NO: 15, and
(ii) a light chain variable region (VL) comprising:
(a) a VL complementarity determining region one (CDR-L1) comprising the amino acid sequence of SEQ ID NO: 8;
(b) a VL complementarity determining region two (CDR-L2) comprising the amino acid sequence of SEQ ID NO: 9; and
(c) a VL complementarity determining region three (CDR-L3) comprising the amino acid sequence of SEQ ID NO: 10.

19. The method as claimed in claim 1, wherein the antibody or antigen binding fragment thereof comprises:
(a) a VH comprising the amino acid sequence of SEQ ID NO: 18, and a VL comprising the amino acid sequence of SEQ ID NO: 11; and/or
(b) a heavy chain comprising the amino acid sequence of SEQ ID NO: 19, and comprises a light chain comprising the amino acid sequence of SEQ ID NO: 12.

20. (canceled)

21. A method for treating hemophilia (e.g., hemophilia A, B or C), comprising administering to a subject in need thereof an initial dose of 300 mg of an antibody or antigen binding fragment thereof that specifically binds to an epitope in Kunitz Domain 2 (K2) of Tissue Factor Pathway Inhibitor (TFPI), followed by administration of a subsequent dose of 150 mg of the antibody or antigen binding fragment thereof, wherein the subsequent dose is administered once a week (weekly) and wherein the antibody comprises (i) a heavy chain comprising the amino acid sequence of SEQ ID NO: 19, and (ii) a light chain comprising the amino acid sequence of SEQ ID NO: 12.

22. A method for treating hemophilia (e.g., hemophilia A, B or C), comprising administering to a subject in need thereof a weekly (once a week) dose of 300 mg of an antibody or antigen binding fragment thereof that specifically binds to an epitope in Kunitz Domain 2 (K2) of Tissue Factor Pathway Inhibitor (TFPI), wherein the antibody comprises (i) a heavy chain comprising the amino acid sequence of SEQ ID NO: 19, and (ii) a light chain comprising the amino acid sequence of SEQ ID NO: 12.

23. (canceled)

24. The method as claimed in claim 1, wherein the subject suffers from or is susceptible to a deficiency in blood coagulation.

25-26. (canceled)

27. The method as claimed in claim 1, wherein administration of the antibody or antigen binding fragment thereof is sufficient to achieve at least 5% of normal hemostatic activity.

28. The method as claimed in claim 1, wherein administration of the antibody or antigen-binding fragment thereof provides a reduction of at least 20% in annualized bleeding rate (ABR) as compared to ABR observed in subjects that have coagulation disorders.

29. The method as claimed in claim 1, further comprising administering a clotting agent to the subject.

30. The method as claimed in claim 1, wherein the clotting agent is selected from the group consisting of factor VIIa, factor VIII, factor IX, tranexamic acid and bypass agent (e.g., FEIBA).

31. A method of reducing annualized bleeding rate (ABR) in a hemophilia subject in need thereof, said method comprising administering a therapeutically effective amount of a TFPI antagonist antibody, wherein the ABR after administration is reduced by at least 80% compared to the ABR in said subject before administration.

32-39. (canceled)

40. A method of reducing annualized bleeding rate (ABR) in a hemophilia subject in need thereof, said method comprising administering a therapeutically effective amount of a TFPI antagonist antibody, wherein the ABR after administration is reduced by at least 85% compared to an ABR historical standard.

41-46. (canceled)
